(19) United States
(12) Patent Application Publication
(10) Pub. No.: US 2024/0382151 A1
(43) Pub. Date: Nov. 21, 2024

(54) MRI-BASED PIPELINE TO EVALUATE RISK OF CONNECTIVE TISSUE REINJURY

(71) Applicants: Children's Medical Center Corporation, Boston, MA (US); RHODE ISLAND HOSPITAL, Providence, RI (US)

(72) Inventors: Ata Kiapour, Wayland, MA (US); Martha M. Murray, Sherborn, MA (US); Braden C. Fleming, Sherborn, MA (US); Sean W. Flannery, Providence, RI (US); Davood Karimi, Brighton, MA (US); Ali Gholipour-Baboli, Westwood, MA (US)

(73) Assignees: Children's Medical Center Corporation, Boston, MA (US); RHODE ISLAND HOSPITAL, Providence, RI (US)

(21) Appl. No.: 18/686,786

(22) PCT Filed: Aug. 26, 2022
(86) PCT No.: PCT/US2022/041691

§ 371 Date: Feb. 26, 2024

Related U.S. Application Data
(60) Provisional application No. 63/237,981, filed on Aug. 27, 2021.

(51) Int. Cl. A61B 5/00; A61B 5/055

(52) U.S. Cl. CPC A61B 5/4585; A61B 5/055; A61B 5/7267

(57) ABSTRACT

Described herein are techniques to aid clinicians and researchers in determining a condition of connective tissue as it relates to tissue development, growth and maturation, tissue remodeling and healing following injury, and risk of injury based on a magnetic resonance (MR) image of the tissue. Such techniques may be useful to clinicians by providing insights on factors that influence the growth and maturation of convective tissues as well as those that impact the risk of connective tissue injury and response to treatment. These insights can be used to guide or develop patient specific risk assessment and prevention strategies, treatment plans, and postoperative care plans for individuals at risk of connective tissue injuries and those with injured connective tissues, such as an anterior cruciate ligament (ACL) injury.

### CROSS-REFERENCE TO RELATED APPLICATIONS

This application claims the benefit under 35 U.S.C. § 119 (e) of U.S. Provisional Patent Application Ser. No. 63/237,981, filed on Aug. 27, 2021, under Attorney Docket No. C1233.70200US00, and entitled “MRI-BASED PIPELINE TO EVALUATE RISK OF CONNECTIVE TISSUE REINJURY,” which is hereby incorporated by reference herein in its entirety.

### GOVERNMENT SUPPORT

This invention was made with government support under Grant No. R01-AR065462awarded by the National Institutes of Health National Institute of Arthritis and Musculoskeletal and Skin Diseases. The government has certain rights in the invention.

### FIELD OF THE DISCLOSURE

The present disclosure relates to techniques to aid clinicians and researchers in determining a condition of connective tissue from magnetic resonance images.

### BACKGROUND

Magnetic resonance imaging (MRI) is a non-invasive and versatile technique for imaging biological systems. Generally, MRI operates by detecting magnetic resonance (MR) signals, which are electromagnetic waves emitted by atoms in response to an applied electromagnetic field. The detected MR signals may then be used to generate MR images of tissues of a patient, usually internal to the patient and unable to be directly viewed without invasive surgery.

### SUMMARY

In one embodiment, there is provided a method of determining a condition of a tissue of a patient, the method comprising: determining a first value indicative of a risk of injury of the tissue from a first magnetic resonance image depicting a tissue; determining a second value indicative of a risk of injury of the tissue from a second magnetic resonance image depicting the tissue and surrounding anatomy; and determining a risk of injury score for the patient using the first value and the second value.

In another embodiment there is provided a computer system, comprising: a computer processor; and a non-transitory computer-readable storage medium storing processor-executable instruction that, when executed by the processor, cause the processor to perform a method of determining a risk for injury of a patient from magnetic resonance image, the method according to any one or any combination of claims 1 to 18.

In another embodiments, there is provided at least one non-transitory computer-readable storage medium storing processor-executable instruction that, when executed by at least one processor, cause the at least one processor to perform the method of determining a condition of a tissue of a patient.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a block diagram of an example of a system for determining a condition of a tissue, in accordance with some embodiments described herein;

FIG. 2 is a flowchart of an illustrative process for determining a condition of a tissue of a patient, in accordance with some embodiments described herein;

FIG. 3 is a flowchart of an illustrative process for segmenting a portion of an image depicting a tissue of interest and the surrounding anatomy, in accordance with some embodiments described herein;

FIG. 4 is a schematic diagram of an illustrative example of a tissue condition determination facility for determining a condition of a tissue of a patient, in accordance with some embodiments described herein;

FIG. 5 illustrates a multi-planar view of an exemplary segmentation of a 3D knee MRI and the resulting segmented ACL, in accordance with some embodiments described herein;

FIG. 6A illustrates a diagram of an exemplary feature extractor facility, in accordance with some embodiments described herein;

FIG. 6B illustrates an exemplary architecture of an Inception-ResNet block, in accordance with some embodiments described herein;

FIG. 7 illustrates an exemplary diagram of an ACL classifier for use in an ACL classification module, in accordance with some embodiments described herein;

FIG. 8 illustrates several exemplary occlusion maps, in accordance with some embodiments described herein;

FIG. 9 illustrates one exemplary implementation of a computing device in the form of a computing device that may be used in a system implementing embodiments of the techniques described herein;

FIG. 10A-C illustrates the performance of an exemplary feature extractor in classifying ACL type from a segmented ACL.

FIGS. 11A-11I illustrates exemplary human performance in classifying ACL type from segmented ACL.

FIG. 12A-12F illustrates exemplary performance of a CNN trained using segmented ACL MRIs in identifying intact and injured ACLs;

FIG. 13A-13C illustrates exemplary performance of a non-imaging classifier in identifying intact and injured ACLs;

FIG. 14A-14I illustrates exemplary performance according to fusions strategies in predicting risk of injury;

FIG. 15A-15I illustrates the classifiers' performance metrics for each independent tested MRI sequence.

### DETAILED DESCRIPTION

Described herein are techniques to aid clinicians and researchers in determining a condition of connective tissue as it relates to tissue development, growth and maturation, tissue remodeling and healing following injury, and risk of injury based on a magnetic resonance (MR) image of the tissue. Such techniques may be useful to clinicians by providing insights on factors that influence the growth and maturation of convective tissues as well as those that impact the risk of connective tissue injury and response to treatment. These insights can be used to guide or develop patient specific risk assessment and prevention strategies, treatment plans, and postoperative care plans for individuals at risk of connective tissue injuries and those with injured connective tissues, such as an anterior cruciate ligament (ACL) injury. Some techniques described herein utilize a magnetic resonance image-based approach to evaluate tissue structure, which may include methods for quantifying connective tissue healing and determining location specific conditions of a tissue that may correspond to a quantitative score indicative of an overall condition of the tissue. This may include assessing location specific conditions of a tissue from an MR image. More particularly, in some embodiments, the methods include determining the condition of tissue and the spatial variation of the condition across the tissue for determining the status of tissue health, strength, remodeling, risk of injury and/or recovery after injury and treatment for a patient. As discussed in more detail below, some techniques described herein include determining a condition of a tissue from an image depicting an isolated tissue and an image depicting a tissue with surrounding anatomy (e.g., surrounding tissue(s)). With some such techniques, initial risk of injury scores may be generated using separate trained classifiers for each image. In some cases, an additional initial risk of injury score may be determined from analysis by another trained classifier of clinical information, which may be non-image information relating to the patient or a clinician's observations of the patient. These initial scores may then be additionally analyzed by another trained classifier, which may output an overall indication of risk of injury to the tissue. The overall indication of risk output by the method may be useful in determining a status of tissue health, strength, remodeling, and/or recovery after injury and treatment for a patient. This approach of analyzing disparate information using disparate classifiers, followed by additional analysis by another classifier of the results of the initial analyses, offers distinct advantages and increased accuracy over conventional approaches, as described below.

Traumatic injuries of joint connective tissue are among the most common musculoskeletal injuries in adolescents and adults participating in physically demanding activities such as sports and military operations. There are more than 17 million joint sprain and strain injuries treated in the U.S. annually. These injuries are often immediately disabling, expensive to treat, and may lead to lasting complications, such as joint degeneration. For example, rupturing of the ACL is one of the most common and devastating connective tissue injuries, primarily occurring to young, active individuals these injuries result in more than 400,000 ACL surgeries each year in the U.S. alone. In many cases, the rate of ACL reinjuries following surgery can be up to 40%, due in part to a lack of effective clinical assessments that have the appropriate sensitivity and specificity to determine when a patient is at a high risk of reinjury.

Accordingly, following an ACL surgery, a major challenge in designing a patient's care plan is determining what types of activities are safe to engage in during various periods of the healing process without risking a reinjury of the ACL. Contemporary clinical assessments for evaluating the ACL during the healing process include clinical examinations (e.g., range of motion), functional tests (e.g., balance); and patient reported outcomes. These assessments may be influenced by factors unrelated to the healing ACL and are prone to observer bias. As such, despite these assessments being widely used, they often fail to reduce the risk of ACL reinjury, due to the low sensitivity and lack of specificity associated with the clinical assessments. The high reinjury rate is due, in part, to the difficulty of assessing the extent to which an ACL has healed following a reconstructive surgery. Without having a precise determination of the health of the tissue, it is challenging to determine the risk of reinjury associated with patient activity. For example, development of an effective patient-specific post-operative care plan, including approving a return to sport, following an ACL surgery is one of the most complex decisions that may be made by a sports medicine team.

Direct assessment of ACL tissue quality could mitigate these shortcomings by providing high sensitivity and patient specific results. The traditional methods of direct assessment used in preclinical studies include comparing biomechanical and histological outcomes. However, due to the destructive nature of these techniques, they are not suitable for clinical studies. A noninvasive, nondestructive technique that allows direct assessment of the ACL could significantly improve post-operative patient outcomes.

MRI is one such noninvasive, nondestructive technique that if used to evaluate the condition of tissue of the ACL could provide advantages over the existing clinical assessments. MR imaging may be used to generate 2D or 3D images of a patient's knee, providing a clinician with a representation of the morphological structure of the tissue. In cases of severe damage, a rupture may be diagnosed from the morphological features of the tissue, such as swelling, changes in the shape, or even orientation of the ACL may be indicative of the damage.

Similarly, some health conditions of the tissue may appear as different intensities in an MR image. In the case of severely damaged tissue, the intensity discrepancies between the healthy tissue and unhealthy tissue may be obvious, and thus may be used to diagnose an injury. However, despite MRI sometimes being used as a technique for diagnosing a tear in an ACL, it is not straightforward to identify a condition, such as the strength, tissue remodeling, healing, or response to treatment of recovering tissue from an MR image. This is due, in part, to the intensity differences, in a recovering ACL, between healthy tissue and unhealthy tissue may be less pronounced and difficult to identify, while morphological features may have no distinguishing characteristics between heathy and unhealthy tissue. Further, the complex differences and variation in morphological and structural features of a healing tissue are extremely challenging to be seen in MRI. As such, the complex analysis required to analyze MR images requires specialized training and may be time intensive.

Thus far, MR imaging has not conventionally been used to predict long term clinical and functional outcomes following ACL surgery. Several studies have tried to use MRI to track changes in ACL health, however, they have not been successful. Such earlier approaches to use MRI to predict long term clinical and functional outcomes have relied on a global measure of the ACL quality, by looking at the whole tissue. As a result, such techniques are insensitive to the distribution of healthy and unhealthy tissue within the ACL and therefore suffer from low accuracy and thus have been unsuitable for determining long term patient outcomes. More specifically, conventional methods that use MR images to determine risk of reinjury evaluate the average intensity corresponding to ACL tissue, and use that average intensity to determine the health of the ACL tissue. Because this analysis of “average” intensity or other global measure fails to account for the distribution of healthy and unhealthy tissue, it can overlook localized weaknesses in the tissue which can lead to reinjuries. For example, a first patient ACL may have several regions of inferior tissue quality distributed around regions of superior tissue quality, while a second patient ACL may have one large region of inferior tissue quality while the rest of the ACL may be superior tissue quality. These two images may reflect the same average intensity but may have drastically different responses to treatment and risks of reinjury. As a result, analysis of signal distribution and its complex pattern may enable more accurate determination of status of tissue health, strength, remodeling, risk of injury, and/or recovery after injury and treatment for a patient. The complex distribution patterns of tissue (e.g., ACL) MRI signals are not captured in available techniques and it is very challenging if not impossible to be understood by the naked human eye. The detailed investigation and quantification of tissue signal and/or relaxation value can help address limitations associated with current techniques which mainly focus on local (i.e., discrete regions of interests) or global (i.e., overall tissue average) values of the signal or relaxation value.

Despite the high prevalence of connective tissue injuries, and despite the substantial flaws and low accuracy of conventional techniques, both without MRI and with MRI, for determining risk of reinjury, those conventional techniques continue to be used. Moreover, the low sensitivity and accuracy of current MRI-based techniques to assess tissue healing and predict risk of injury have limited their clinical utility.

The inventors have recognized and appreciated that such challenges may be mitigated by using machine learning approaches (e.g., deep learning) to determining the condition of the tissue at multiple points of the tissue in the MR image and examining the distribution of signal and/or relaxation values across the tissue depicted in the MR image. Some distributions of the condition of the tissue may result in a higher risk of reinjury than others. In particular, the MR image depicting the ACL tissue may be evaluated by one or more trained classifiers to determine a value indicative of a risk of injury.

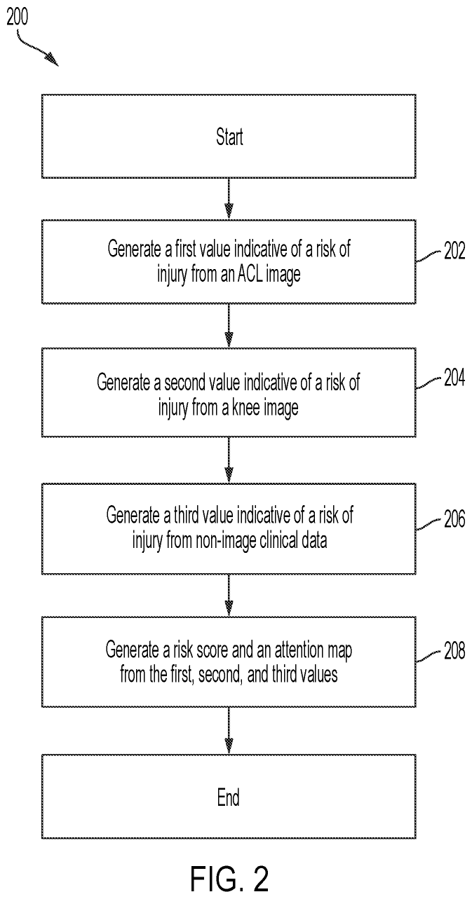

The inventors have accordingly developed systems and methods, described herein, to determine a condition of a tissue of a patient based on a MR image of the tissue, including techniques using machine learning algorithms. In some embodiments, determining a risk of injury includes an image-based approach to guide postoperative care of patients with connective tissue injury by determining a risk of injury score from both an image depicting the connective tissue(s) in isolation and an image depicting the connective tissue with the surrounding anatomy. The inventors have realized that surrounding anatomy is customarily not analyzed and is instead screened from analysis, as data relating to surrounding anatomy can interfere with an evaluation of the connective tissue(s) resulting in a decrease in the accuracy of the determined risk score. However, the inventors have recognized that analyzing the tissue(s) in isolation from the surrounding anatomy may not account for the effects that the condition of the surrounding anatomy can have on the tissue(s), which also affects an accuracy of the risk score. According to some embodiments described herein, both images (the image depicting the connective tissue(s) and the image depicting the connective tissue with the surrounding anatomy) are analyzed to develop individual assessments of risk of injury. More particularly, in some embodiments, an analysis is performed of an image of the tissue(s) of interest and another analysis is performed of an image of the tissue(s) and the surrounding anatomy. After the initial assessments of risk of injury are determined, the results of those initial assessments are further analyzed to yield a risk score that is output as a risk of injury to the tissue(s). Such a combined approach yields an increased accuracy relative to the analysis from either of the individual images. In some embodiments, non-image information (e.g., demographics, lifestyle and clinical and functional outcomes) may be additionally analyzed, such as by generating an initial risk score that is subsequently also analyzed with the initial scores from the analysis of images to generate the output risk score. Aspects of the image-based approach to guide postoperative care of patients with connective tissue injury are described herein including with reference to FIGS. 2-4.

Accordingly, some embodiments provide a method of determining a condition of a tissue (or one or more tissues) of a patient, the method includes determining a first value indicative of a risk of injury of the tissue from a first magnetic resonance image depicting tissue and determining a second value indicative of a risk of injury of the tissue from a second magnetic resonance image depicting the tissue and the surrounding anatomy. As one illustrative example, the depicted tissue in the first image may be an ACL and the depicted tissue and surrounding anatomy in the second image may be the ACL and the surrounding anatomy of the knee of the patient. Additionally, the method may include determining a risk of injury score for the patient using the first and second values.

In some embodiments, separate trained classifiers are used to determine the first value, the second value, and the risk of injury. For example, a first value may be determined using a first trained classifier from the first image, a second value may be determined using a second trained classifier from the second image, and the risk of injury may be determined using an additional trained classifier which receives the first value and the second value as inputs to determine the risk of injury. In such a case, the first image may depict the tissue in isolation and the second image may depict the tissue and surrounding anatomy. In some embodiments, the first image may have been generated from the second image. For example, the first image may be a segmented portion of the second image, with the segmented portion corresponding to pixels or voxels depicting the tissue.

Additionally, or alternatively, the method may include determining a third value indicative of a risk of injury of the tissue from non-image data. For example, the third value is determined using a third trained classifier from clinical non-image data. The third value may be used with the first and second values to determine a risk of injury score for the patient. For example, the first, second, and third values may be evaluated using the risk of injury classifier to determine an overall risk of injury to the tissue.

In some embodiments, determining a risk score for the patient may also include generating an attention map. The attention map may be a visual representation of the tissue and surrounding tissue with annotations to indicate locations in the representation that correspond to anatomy of a patient that contribute to an increased risk of injury. For example, the attention map may be an image depicting the tissue and surrounding anatomy with certain locations corresponding to one or more levels of increased risk of injury distinguished by certain visual features, such as different colors, different shading, different outlines, textual labels, or other different visual features for the locations with increased risk (or for different locations with different levels of increased risk) than other locations of the image. The attention map may be used by clinicians to identify critical features and structures contributing to the outcomes of surgery and risk of injury. Such information can be used to develop new anatomic biomarkers to track tissue healing and ultimately to personalize the postoperative care plan accordingly. Furthermore, the attention map may be used to train clinicians in recognizing reading MR images to determine a risk of injury.

Various examples of ways in which these techniques and systems may be implemented are described below. It should be appreciated, however, that embodiments are not limited to operating in accordance with these examples. Other embodiments are possible.

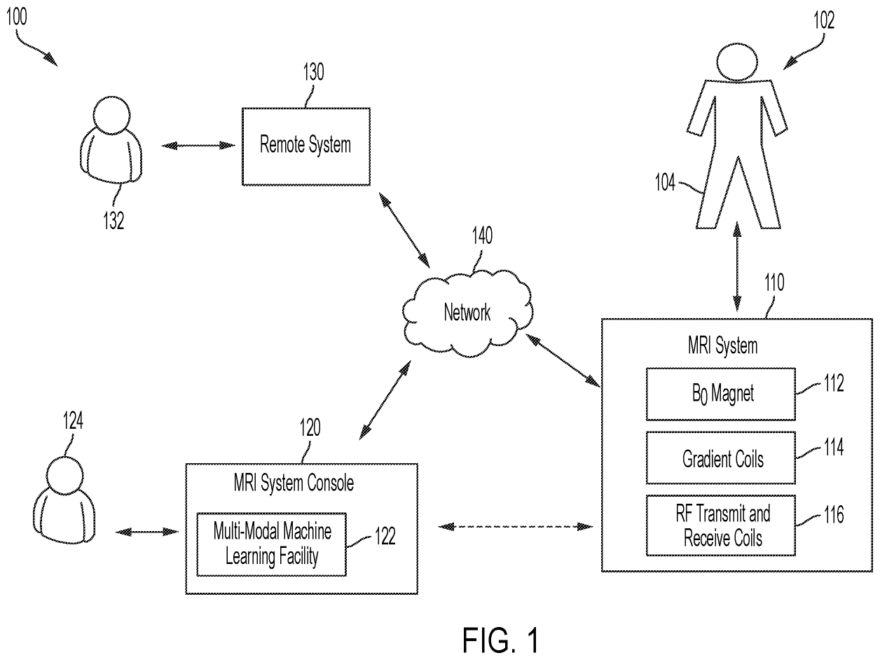

FIG. 1 is a block diagram of an example of a system 100 for determining a condition of a tissue, in accordance with some embodiments described herein. In the illustrative example of FIG. 1, system 100 includes an MRI system 110, and MRI system console 120, and a remote system 130. It should be appreciated that system 100 is illustrative and that a system may have one or more other components of any suitable type in addition to or instead of the components illustrated in FIG. 1. For example, there may be additional remote systems (e.g., two or more) present within a system.

As illustrated in FIG. 1, in some embodiments, one or more of the MRI system 110, the MRI system console 120, and the remote system 130 may be communicatively connected by a network 140. The network 140 may be or include one or more local and/or wide-area, wired and/or wireless networks, including a local-area or wide-area enterprise network and/or the Internet. Accordingly, the network 140 may be, for example, a hard-wired network (e.g., a local area network within a healthcare facility), a wireless network (e.g., connected over Wi-Fi and/or cellular networks), a cloud-based computing network, or any combination thereof. For example, in some embodiments, the MRI system 110 and the MRI system console 120 may be located within the same healthcare facility and connected directly to each other or connected to each other via the network 140, while the remote system 130 may be located in a remote healthcare facility and connected to the MRI system 110 and/or the MRI system console 120 through the network 140.

In some embodiments, the MRI system 110 may be configured to perform MR imaging of anatomy of a patient 102, such as a knee 104 of the patient, in some scenarios. For example, the MRI system 110 may include a BO magnet 112, gradient coils 114, and radio frequency (RF) transmit and receive coils 116 configured to act in concert to perform said MR imaging.

In some embodiments, B0 magnet 112 may be configured to generate the main static magnetic field, B0, during MR imaging. The B0 magnet 112 may be any suitable type of magnet that can generate a static magnetic field for MR imaging. For example, the B0 magnet 112 may include a superconducting magnet, an electromagnet, and/or a permanent magnet. In some embodiments, the B0 magnet 112 may be configured to generate a static magnetic field having a particular field strength.

In some embodiments, gradient coils 114 may be arranged to provide one or more gradient magnetic fields. For example, gradient coils 114 may be arranged to provide gradient magnetic fields along three substantially orthogonal directions (e.g., x, y, and z). The gradient magnetic fields may be configured to, for example, provide spatial encoding of MR signals during MR imaging. Gradient coils 114 may comprise any suitable electromagnetic coils, including discrete wire windings coils and/or laminate panel coils.

In some embodiments, RF transmit and receive coils 116 may be configured to generate RF pulses to induce an oscillating magnetic field, B1, and/or to receive MR signals from nuclear spins within a target region of the imaged subject (e.g., of the knee 104) during MR imaging. The RF transmit coils may be configured to generate any suitable types of RF pulses useful for performing MR imaging. RF transmit and receive coils 116 may comprise any suitable RF coils, including volume coils and/or surface coils.

As illustrated in FIG. 1, system 100 includes MRI system console 120 communicatively coupled to the MRI system 110. MRI system console 120 may be any suitable electronic device configured to send instruction and/or information to MRI system 120, to receive information from MRI system 120, and/or to process obtained MR data. In some embodiments, MRI system console 120 may be a fixed electronic device such as a desktop computer, a rack-mounted computer, or any other suitable fixed electronic device. Alternatively, MRI system console 120 may be a portable device such as a laptop computer, a smart phone, a tablet computer, or any other portable device that may be configured to send instructions and/or information to MRI system 120, to receive information from MRI system 120, and/or to process obtained MR data.

Some embodiments may include a tissue condition determination facility 122. Tissue condition determination facility 122 may be configured to analyze MR data obtained by MRI system 110 from an MR imaging procedure of patient 102. Tissue condition determination facility 122 may be configured to, for example, as a multi-modal machine learning facility to analyze the obtained MR data to determine the condition of a tissue for one or more sets of MR data, as described herein. Tissue condition determination facility 122 may be implemented as hardware, software, or any suitable combination of hardware and software, as aspects of the technology described herein are not limited in this respect.

As illustrated in FIG. 1, the tissue condition determination facility 122 may be implemented in the MRI system console 120, such as by being implemented in software (e.g., executable instructions) executed by one or more processors of the MRI system console 120. However, in other embodiments, the tissue condition determination facility 122 may be additionally or alternatively implemented at one or more other elements of the system 100 of FIG. 1. For example, the tissue condition determination facility 122 may be implemented at or with another device, such as a device located remote from the system 100 and receiving data via the network 140.

MRI system console 120 may be accessed by MRI user 124 in order to control MRI system 120 and/or to process MR data obtained by MRI system 120. For example, MRI user 124 may implement an MRI imaging process by inputting one or more instructions into MRI system console 120 (e.g., MRI user 124 may select an MR imaging process from among several options presented by MRI system console 120). Alternatively, or additionally, in some embodiments, MRI user 124 may implement an MR data analysis procedure by inputting one or more instructions into MRI system console 120 (e.g., MRI user may select MR data instances to be analyzed by MRI system console 120).

As illustrated in FIG. 1, MRI system console 120 also interacts with remote system 130 through network 140, in some embodiments. Remote system 130 may be any suitable electronic device configured to receive information (e.g., from MRI system 110 and/or MRI system console 120) and to display generated MR images for viewing. The remote system 130 may be remote from the MRI system 110 and MRI system console 120, such as by being located in a different room, wing, or building of a facility (e.g., a healthcare facility) than the MRI system 110, or being geographically remote from the system 110 and console 120, such as being located in another part of a city, another city, another state or country, etc. In some embodiments, remote systems 130 may be a fixed electronic device such as a desktop computer, a rack-mounted computer, or any other suitable fixed electronic device. Alternatively, remote system 130 may be a portable device such as a laptop computer, a smart phone, a tablet computer, or any other portable device that may be configured to receive and view generated MR images and/or to send instructions and/or information to MRI system console 120.

In some embodiments, remote system 130 may receive information (e.g., MR data analysis results, generated MR images of a patient, and/or raw MR data) from MRI system console 120 and/or MRI system 110 over the network 140. A remote user 132 (e.g., the patient's medical clinician) may use remote system 130 to view the received information on remote system 130. For example, the remote user 132 may view generated MR images using remote system 130 after the MRI user 124 has completed MR data analysis using MRI system 110 and/or MRI system console 120.

The inventors have recognized and appreciated that a tissue condition determination facility that uses separate classifiers, based on different inputs, for determining the condition of a tissue could enable accurate, sensitive, and specific determination of condition of a tissue. Such a tissue condition determination facility may be used in connection with the system 100 illustrated in FIG. 1 for determining a condition of a tissue, status of tissue health, the strength of ligament tissue, remodeling of the tissue, risk of injury, and/or recovery after injury and treatment for a patient.

The inventors have further recognized that the variations in MR images produced using different MR imaging sequences and/or acquisition parameters present a challenge to implementing the tissue condition determination facility in a clinic setting. The characteristics of MR images (i.e., how structural features appear in the MR image) depend on the imaging sequence and acquisition parameters. Accordingly, a facility configured to process MR images produced using one MR imaging sequence may not similarly be configured to process MR images produced using other MR imaging sequences or acquisition parameters, which can limit the generalizability and clinical implementation of methods for analyzing MR images. Therefore, the inventors have developed a tissue condition determination facility that may classify the risk of injury of ACLs independently of the MRI sequence used for imaging.

FIG. 2 is a flowchart of an illustrative process 200 for determining a condition of a tissue of a patient, in accordance with some embodiments. Prior to the start of process 200, the tissue condition determination facility may receive MR image data corresponding to an imaging of a patient. In some embodiments, the tissue condition determination facility may interface with an MRI system to receive the MR image data. In some embodiments, the tissue condition determination facility may interface with an MRI system to instruct the MRI system to acquire an MR image. For example, the tissue condition determination facility may provide parameters related to the MR imaging sequence. Following the acquisition of the MR image, the tissue condition determination facility may receive the resulting MR image data.

Additionally, prior to the start of process 200 a 3D MR image depicting a patient's knee (e.g., a full knee image) may be segmented such that a 3D image depicting the ACL without the surrounding tissue (e.g., bone and other ligaments) is generated. In some embodiments, the 3D image depicting the ACL and the 3D MR image depicting the patient's knee may both be provided as inputs to process 200. In some embodiments, clinical data associated with the patient depicted in the MR image may be provided and/or accessible to the tissue condition determination facility, such that the clinical data may be provided as an additional input to process 200.

Process 200 begins at block 202, the tissue condition determination facility generates a first value indicative of a risk of injury from an ACL image. The ACL image may be a segmented image of a full knee MR image such that the segmented image depicts the ACL without the surrounding tissue. In some embodiments, the tissue condition determination facility determines a risk of injury based on the spatial variation of healthy tissue of the ligament. To evaluate the spatial variation of the healthy tissue, a tissue pattern may be determined. The tissue pattern may include the size of healthy and/or damaged regions of tissue; the shape of healthy and/or damaged tissue regions of tissue; the spacing between regions of healthy and/or damaged tissue; and/or other parameters for characterizing the tissue patterns. For example, the tissue condition determination facility may compare the 3D variation of healthy and/or damaged tissue from a patient's ACL with patterns that that the tissue condition determination facility associates with a healthy ACL (i.e., decreased risk of experiencing an ACL injury) and damaged ACL (i.e., increased risk of experiencing an ACL injury) to determine a value indicative of a risk of injury (i.e., a risk of injury sub-score) for the patients ACL.

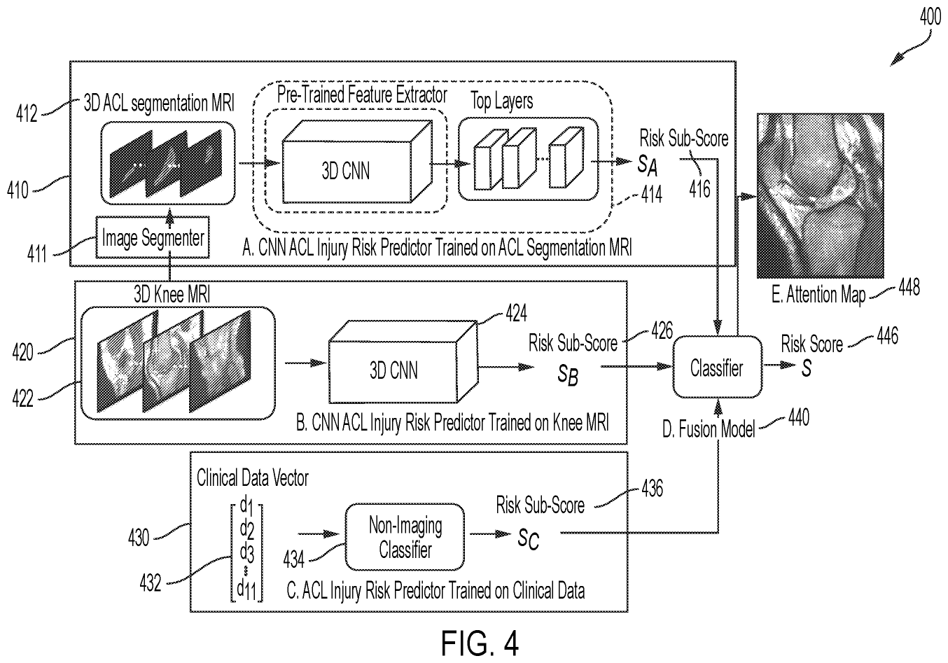

In some embodiments, the tissue condition determination facility may use a first classifier trained to determine a first value indicative of a risk of injury based on ACL MR images. For example, the first classifier may be a first convolution neural network (CNN) trained on ACL MR images to determine a risk of injury. In other embodiments, other classifiers may be used, or other analysis techniques may be used (e.g., that do not include a classifier), as aspects of the technology described herein are not limited in this respect. In some embodiments, the first classifier may be ACL classification module 410, as described further below in connection with FIG. 4.

At block 204, the tissue condition determination facility generates a second value indicative of a risk of injury from a knee image. The knee image may depict the ACL tissue that was depicted in the ACL image processed in block 202 as well as surrounding tissue(s). The knee image may be the same image from which the ACL image was segmented as an input to the first CNN classifier to determine the first value at block 202, or a different image of that knee, as embodiments are not limited in this respect. In some embodiments, the tissue condition determination facility determines a risk of injury based on the spatial variation of healthy tissue of the ligament and the surrounding tissue. In addition to determining a tissue pattern, the location of healthy and/or damaged tissue relative to the surrounding tissue may be determined. The location of healthy and/or damaged tissue relative to the surrounding tissue may impact the risk of injury associated with a portion of damaged tissue. For example, some portion of the ligament tissue may be subjected to increased stress during use, relative to other portions of the ligament tissue. Thus, when a portion of the ligament which is subject to increased stress includes damaged tissue, the risk of injury may be increased. The increased stress during use, on some portions of the ligament tissue, may be impacted by the surrounding tissue. Therefore, in some embodiments, the spatial variation of healthy and/or damaged tissue may be weighted differently, based on its position, in determining a value indicative of a risk of injury.

In some embodiments, the tissue condition determination facility may use a second classifier to determine a second value indicative of a risk of injury of the ACL based on the knee MR image. For example, the second classifier may be a second CNN trained on full knee MR images to determine a risk of injury. In other embodiments, other classifiers may be used, as aspects of the technology described herein are not limited in this respect. In some embodiments, the second classifier may be full knee classification module 420, as described further below in connection with FIG. 4.

At block 206, the tissue condition determination facility generates a third value indicative of a risk of injury from non-image clinical data. In some embodiments, non-image clinical data includes results from a physician's examination of the knee. The non-image clinical data may additionally include aspects of a patient's medical history and/or self-reported outcomes (e.g., questionnaire responses). The non-image clinical data, associated with the patient depicted in the knee and ACL images, may be input as a vector of input values corresponding to different clinical parameters into a third classifier. In some embodiments, the vector may include 11 different input values, as described herein. Other input vectors including a different number of the same and/or different clinical parameters may also be used, as aspects of the technology described herein are not limited in this respect.

In some embodiments, the third classifier may use a logistic regression-based model trained to determine a third value indicative of a risk of injury based on non-imaging clinical data. In some embodiments, the third classifier may be non-imaging classification module 430, as described further below in connection with FIG. 4.

In some embodiments, other machine learning models may be used in determining the first, second, and/or third values indicative of a risk of injury, as aspects of the technology described herein are not limited in this respect.

At block 208, the tissue condition determination facility generates a risk score from the first, second, and third values indicative of a risk of injury and as generated at blocks 202, 204, and 206. In some embodiments, a fourth classifier is used to determine a risk score based on the first, second, and/or third values. In some embodiments, the fourth classifier may be a late-stage fusion module trained to determine risk of injury based on the first. second, and third values indicative of the risk of injury. For example, the late-stage fusion module may be a Logistic Regression classifier configured to determine a probably of injury between as a binary dependent variable of the three values indicative of the risk of injury. In other embodiments, other classifiers may be used, as aspects of the technology described herein are not limited in this respect. In some embodiments, the fourth classifier may be risk classifier 440, as described further below in connection with FIG. 4.

In some embodiments, the tissue condition determination facility also generates an attention map, indicating portions of the tissue(s) depicted in the MR image(s) that contributed to increasing the risk of injury. A color overlay may be used where the color, pattern, and/or intensity may be used to indicate the degree with which a portion of the depicted tissue contributes to the risk of injury.

After the risk score is generated at block 208, process 200 ends. The risk score may be used by a clinical care team to tailor the postoperative care regimen, including the rehabilitation strategy and/or return to sports plan based. Although, the clinical care team is in charge of ultimate decision, the tissue condition determination facility can provide additional insights to facilitate the decision-making process, offering improvements over the low sensitivity of current clinical tools.

The tissue condition determination facility may be used to screen cases, in an automated manner with minimum human involvement, to flag the cases with high risk of subsequent injury. Flagged cases can then be prioritized in clinical workflows to receive additional attention or even a second opinion. Additionally, the inventors have appreciated that the tissue condition determination facility can assist with the predication of long-term ACL surgery outcomes, in particular the risk of knee osteoarthritis (OA). The inventors have further appreciated that, the detailed feature extractor and occlusion maps can assist with the identification of new imaging biomarkers and image scoring criteria to better track the healing of a surgically treated ACL. Such feedback to track the healing of an ACL may help in evaluating new treatments (e.g., Bridge-Enhanced ACL Repair), and assisting with obtaining regulatory approvals, as different treatments may result in different structural features compared to current ACL treatments (e.g., ACL reconstruction).

The inventors have recognized that a further challenge to implementing the tissue condition determination facility in a clinic setting is the pre-processing required to manually segment the ACL, as a primary input to the model. Therefore, the inventors have developed a segmentation facility that may be used in some embodiments, in connection with method 200, to segment the portion of a 3D image depicting a patient's knee that corresponds to the ACL. Other segmentation techniques may be used in other embodiments.

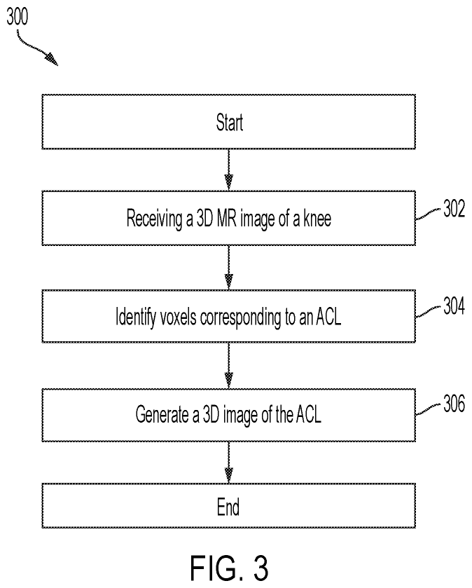

FIG. 3 is a flowchart of an illustrative process 300 for segmenting a portion of an image depicting a tissue of interest and the surrounding anatomy, in accordance with some embodiments. Process 300 begins at block 302 where the tissue condition determination facility receives a 3D MR image depicting a tissue of interest of a patient and the surrounding tissue. In some embodiments, the tissue of interest is a ligament. For example, the tissue of interest may be the ACL. In some embodiments, the 3D MR image is an image of a knee including the ACL in the field of view. The MR image may be acquired according to any MR sequence, as described herein.

In some embodiments, the 3D MR image may be a stack of 2D images representing 2D slices through the knee. In other embodiments, the 3D MR image may be a point cloud including the intensity and/or phase of the MR signal at points corresponding to the spatial position of the MR signal. Other 3D image formats may be used, as aspects of the technology described herein are not limited in this respect.

Next, at block 304, the tissue condition determination facility identifies voxels that correspond to the tissue of interest. In some embodiments, the tissue condition determination facility identifies voxels in a 3D MR image of the knee that correspond to an ACL. In some embodiments, a machine learning module, such as a CNN, may be used to identify the voxels in the 3D MR image of the knee that correspond to the ACL. In other embodiments, identifying voxels in the 3D MR image of the knee that correspond to the ACL may involve thresholding, edge-detection, and/or clustering methods. In yet other embodiments, input from a user may be used to identify voxels in the 3D MR image. Other techniques for identifying voxels in the 3D MR image of the knee that correspond to the ACL are possible, as aspects of the technology described herein are not limited in this respect.

Next at block 306, the tissue condition determination facility generates a 3D image of the tissue of interest. In some embodiments, the tissue condition determination facility generates a 3D image of the ACL using the voxels identified at block 304 as including the ACL. The 3D image of the ACL may be reproduced in a separate image, such that the separate image depicts the ACL without the surrounding tissue (e.g., bone and other ligaments). In other embodiments, a mask may be generated such that, when the mask is applied, the portions of the image depicting the ACL are visible while the portions of the image depicting the surrounding tissue are obscured by the mask.

After the tissue condition determination facility generates a 3D image of the ACL, process 300 is complete. Following the completions of process 300, the 3D image of the ACL may be transmitted as an input to an ACL classifier, trained to generate a value indicative of a risk of injury from an ACL image. Additionally, or alternatively, the MR image of the knee, from which the segmented ACL image was generated, may be sent as an additional input to a classifier trained to generate a value indicative of a risk of injury from a full knee MR image.

As discussed above, the inventors have recognized and appreciated that a tissue condition determination facility may mitigate or address the challenges of predicting the risk of ACL injury after surgical treatment. In some embodiments, a deep learning approach can achieve high performance in classifying cases who had a subsequent ACL injury after ACL surgery. For some applications, the deep learning facility has a superior performance, in extracting ACL features that may be used to classify ligament type, compared to experienced human examiners. The inventors have recognized that analyzing isolated ACLs, segmented from a whole knee MRI, may provide advantages determining a risk score when compared to the same analysis based on full knee MRIs and/or non-imaging outcomes. The inventors have further recognized that using a combination of isolated segmented ACLs, whole knee MRI and non-imaging clinical information may provide further advantages, relative to each respective analysis.

FIG. 4 is a schematic diagram of an illustrative example of a tissue condition determination facility 400 for determining a condition of a tissue of a patient, in accordance with some embodiments. As illustrated in FIG. 4, the tissue condition determination facility may include ACL classification module 410, full knee classification module 420, and non-imaging classification module 430. The classification modules produce risk sub-scores which are input into risk classifier 440. Risk classifier 440 may be implemented using a late-stage fusion module that uses the risk sub-scores to determine a risk score 446, indicative of the risk of ACL injury. Additionally, the risk classifier 440 may produce an attention map 448 that illustrates a portion(s) of tissue that contribute(s) to the risk of injury. In some embodiments, tissue condition determination facility 400 may be configured to implement processes 200 and/or 300, as described above in connection with FIGS. 2 and 3.

ACL classification module 410 includes image segmenter 411 for segmenting a portion of a full knee MRI a to produce a segmented ACL MR image 412, in accordance with some embodiments. The segmented ACL MR image 412 may be used as an input to an ACL classifier 414 to produce output risk sub-score 416, in accordance with some embodiments. The segmented ACL MR image 412 used as an input to the ACL classifier 414. The segmented ACL MR image 412 may be segmented from a full knee MR image 422. The ACL classifier 414 may be implemented using a CNN trained using segmented ACL MR images. The training of ACL classifier 414 may include a pre-trained feature extractor, trained to identify type of tissue and top layers for determining a risk sub-score. In some embodiments, the pre-trained feature extractor may include one or more frozen layers.

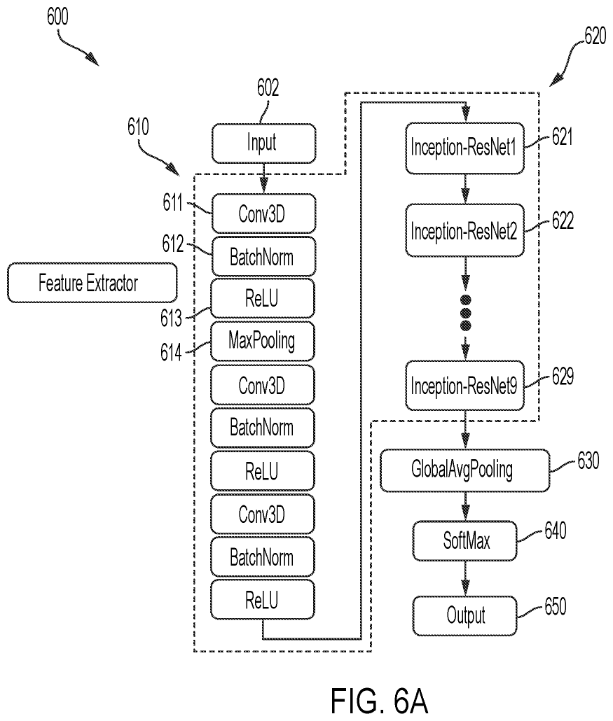

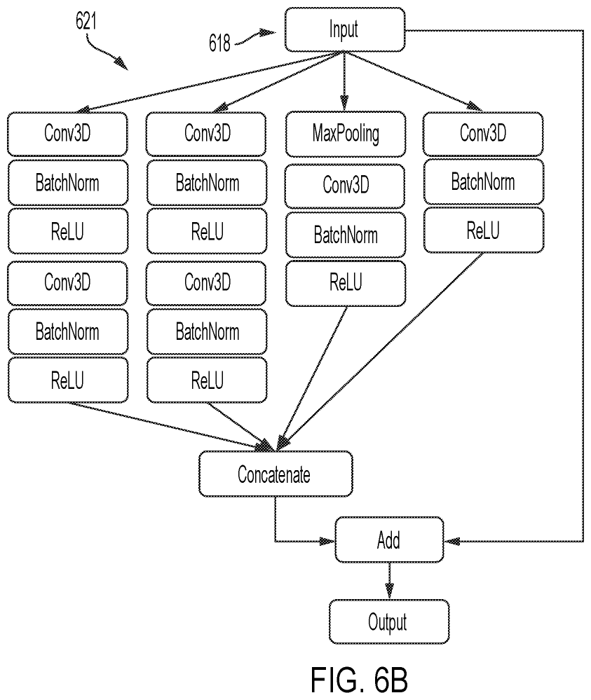

The risk sub-score 416 may indicate a probability of injury occurring to the ACL, in accordance with some embodiments. The pre-trained feature extractor may identify the type of tissue as native, bridge-enhanced ACL repair (BEAR), or ACL reconstruction (ACLR) as described further below in connection with FIG. 6A-6B.

Full knee classification module 420 may receive a full knee MR image 422 as an input to a full knee classifier 424 to produce output risk sub-score 426, in accordance with some embodiments. The full knee MR image 422, may be the same image which is used to produce the segmented ACL MR image 412. The full knee classifier 424 may be implemented using a CNN trained using full knee MR images. The training may be implemented using a pre-trained feature extractor trained to identify type of tissue and top layers for determining a risk sub-score, as described further below in connection with FIG. 6A and 6B.

In addition to the MR images, patient demographics (e.g., age and sex) and/or clinical outcomes that evaluate ACL injury and recovery may be evaluated by a classifier to determine a condition of an ACL tissue. Clinical outcomes may include patient reported outcomes where a patient performs a self-assessment of knee function. A patient reported income may include a patient evaluated pain, stiffness, swelling of their knee and/or other assessments. For example, a patient may determine an International Knee Documentation Committee Subjective score (IKDC score), which may be evaluated by a classifier to determine risk of injury. Additionally, or alternatively, other clinical outcomes, such as, functional assessments may be performed by a clinician to indicate the clinician's assessment of knee function. For example, a clinician may perform a functional assessment focused on knee laxity, muscle strength, single legged hop performance, and/or knee range of motion. The results of the functional assessment may be evaluated by a classifier to determine a risk of injury.

Non-imaging classification module 430 may receive clinical data as an input to non-imaging classifier 434 to produce risk sub-score 436, in accordance with some embodiments. The non-imaging outcomes measured in a clinical setting may be input as a clinical data vector 432 with 11 parameters. The parameters may include, IKDC scores, instrumented AP Knee Laxity assessment scores, muscle strength assessment scores, knee range of motion scores, and functional hop test scores, examples of which are further detailed below. In other embodiments, the clinical data vector 432 may have a different number of parameters, including a sub-set of the 11 parameters, or additional clinical parameters, as aspects of the technology described herein are not limited in this respect. The non-imaging classifier 434 may be implemented using a logistic regression classifier, trained to distinguish between intact and injured ACLs based on non-imaging outcomes, and may provide a non-imaging-based risk of injury sub-score for the tissue condition determination facility, in accordance with some embodiments.

Additionally, or alternatively, a Naive Bayes, Nearest Neighbor, Random Forest, Linear Support Vector Machine (SVM), Linear Discriminant Analysis (LDA), Decision Tree, and/or other classifiers may be used to provide the non-imaging-based risk of injury sub-score for the tissue condition determination facility, in accordance with some embodiments.

For example, non-imaging classifier 430 may integrate the potentially relevant non-imaging clinical data in predicting the risk of ACL injury. The non-imaging input may be an 11-dimensional vector of variables. Each value in the data vector may be associated with a binary label that indicates Intact/Injured ACL. The non-imaging classifier outputs an estimated probability of an Intact verses an Injured ACL. The predicted probability of the Injured ACL class may be used as the ACL injury risk sub-score. In other embodiments, other classifiers may be used in addition to or as an alternate to the logistic regression classifier, as aspects of the technology described herein are not limited in this respect.

The risk sub-scores produced by the ACL classification module 410, full knee classification module 420, and non-imaging classification module 430 may be used by the risk classifier 440 to determine the risk score 446. In some embodiments, the risk classifier 440 may use a fusion module to determine the risk score 446 based on a single sub-score. In other embodiments, the risk classifier 440 may use a fusion module determine the risk score 446 based on a combination of two of the sub-scores. In yet other embodiments, the risk classifier may use a fusion module to determine the risk score 446 based on a combination of the three sub-scores.

Additionally, or alternatively, a Naive Bayes, Nearest Neighbor, Random Forest, Linear Support Vector Machine (SVM), Linear Discriminant Analysis (LDA), Decision Tree, and/or other classifiers may be used by the risk classifier 440 to determine the risk score 446, in accordance with some embodiments.

As discussed above, the ACL classification module may receive an ACL MR image, as an input, that is segmented from a full knee MRI image, where the full knee MRI image is used as an input to the full knee classification module, in accordance with some embodiments. A segmentation module may be used to produce the segmented ACL MR image by extracting voxels from a 3D knee MR image such that the segmented MR image includes voxels that correspond to ACL tissue without including voxels corresponding to the bone or other ligaments also present in the full knee MR image. The segmentation module may be implemented using process 300, as described above in connection with FIG. 3.

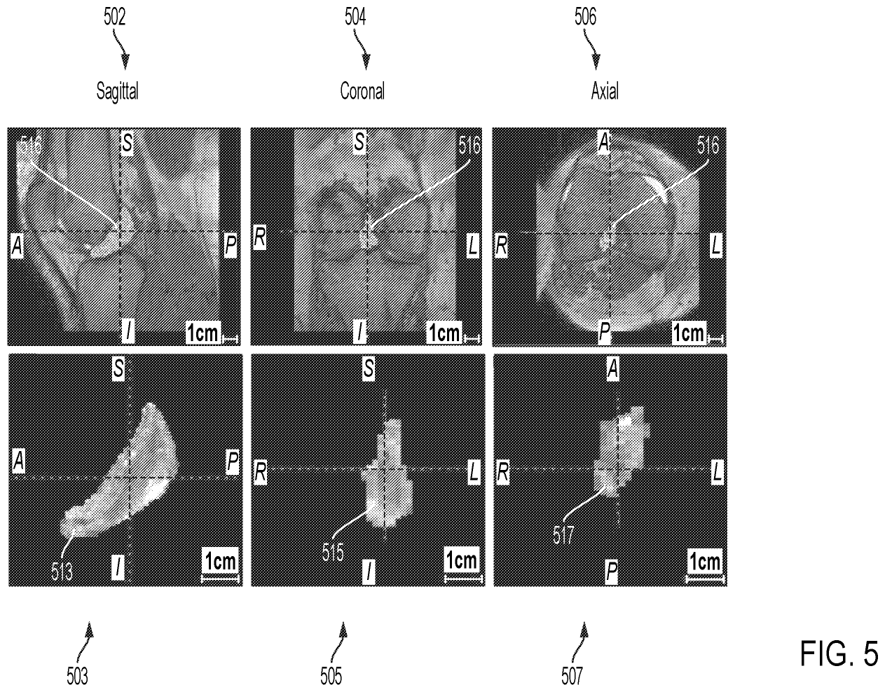

FIG. 5 illustrates a multi-planar view of an exemplary segmentation of a 3D knee MRI and the resulting segmented ACL, in accordance with some embodiments. MR image 502 illustrates a sagittal view of a 3D knee MRI with the ACL portion removed from image area 512. MR image 503 illustrates the segmented portion of the ACL 513 removed from the sagittal view of MR image 502. MR image 504 illustrates a coronal view of a 3D knee MRI with the ACL portion removed from image area 514. MR image 505 illustrates the segmented portion of the ACL 515 removed from the coronal view of MR image 504. MR image 506 illustrates an axial view of a 3D knee MRI with the ACL portion removed from image area 516. MR image 507 illustrates the segmented portion of the ACL 517 removed from the axial view of MR image 506.

The inventors developed and trained separate deep CNNs, to implement the ACL classification module trained on isolated ACL MR images and the full knee classification module trained on full knee MR images of the tissue condition determination facility. In some embodiments, to identify detailed features of the ACL and their differences between native and surgically treated tissues the base layers of a CNN may be implemented using a feature extractor facility, that includes 3D Inception-ResNet architecture, trained towards discriminating the type of ligament (native, repaired and reconstructed) from isolated ACL MR images.

FIG. 6A illustrates a diagram of an exemplary feature extractor facility 600, in accordance with some embodiments. The feature extractor facility 600 may be implemented using a deep CNN to identify detailed features of the ACL and how those features differ between native and surgically treated tissues. The feature extractor facility 600 includes stem 610 and residual network (ResNet) blocks 620. Stem 610 includes convolution (Conv) layer 611, pooling layer 614, normalization (BatchNorm) layer 612, and rectified linear activation function (ReLu) layer 613 that are applied to a received input 602 prior to applying ResNet blocks 620. Global average pooling 630 and SoftMax 640 layers may be applied to the result from the ResNet blocks 620 to generate output 650.

ResNet blocks 620 may include nine Inception-ResNet blocks, according to some embodiments. For example, ResNet blocks 620 may include a first Inception-ResNet block 621 and a second Inception-ResNet block 622 configured such that the output of the first Inception-ResNet block 621 is input into the second Inception Res-Net block 622. The ResNet blocks 620 may further include additional Inception-ResNet blocks arranged sequentially such that each block receives an input from the preceding block until a final Inception-ResNet block 629 which provides its output to a pooling layer such as GlobalAvgPooling layer 630. In other embodiments, other numbers of ResNet blocks may be used, as aspects of the technology described herein are not limited in this respect. For example, between one and nine Inception ResNet blocks may be used. As another example, greater than nine Inception ResNet blocks may be used. The Inception-ResNet blocks may include 3D convolutional layers, in accordance with some embodiments.

The input 602, received by the CNN feature extractor facility, may be a masked portion of an ACL segmented from a knee MR image, in accordance with some embodiments. For example, the CNN feature extractor facility 600 may be configured to receive segmented ACL masks of size 128×128×64 voxels as input. Other mask sizes may be used, as aspects of the technology described herein are not limited in this respect.

The output of global averaging is passed through a softmax layer to obtain the conditional class probability estimates, q(yᵢ|x), where x is the input MR image and yᵢ are the set of labels used to represent the three classes of native, BEAR and ACLR. In some embodiments, the CNN feature extractor is optimized through a back-propagation algorithm to minimize the cross-entropy loss between the estimated and true class probability vectors. In some embodiments, the loss function may be defined using the following equation:

$$- {𝔼}_{p} [ log {q}_{γ} ( y \vert x ) ] = - {\sum }_{i = 1}^{3} p ( {y}_{i} ) {q}_{γ} ( {y}_{i} \vert x )$$

where p(yᵢ) and qᵣ(yᵢ|x) are, respectively, the true and predicted probabilities of class yᵢ, and γ denotes the set of all model parameters to be optimized. In some embodiments, the optimization is performed using the Adam optimizer with a learning rate of 10⁻⁵.

The inventors have appreciated that implementing CNNs for 3D image analysis provides challenges. CNNs are usually implemented in 2D image analysis, however, because of the large number of convolutional operations involved, especially for large 3D images, 3D CNN modules can have a prohibitively large memory footprint. Therefore, in order to reduce the GPU memory requirements, the inventors have developed a CNN architecture that first applies a series of 3D convolutional layers followed by a pooling layer to reduce the size of input data while preserving features relevant to the image analysis. Pooling layers may combine data values together through downsampling. For example, for a pool layer with a size of 2×2, corresponding to a matrix with two rows and two columns to produce which can include four values, input data of a larger size will be divided up into quadrants and then each quadrant is used to determine a single value for the pool layer. In some pool layers, the maximum value from the quadrant is used as the value for the pool layer (i.e., max pooling). In other pool layers, the average value from the quadrant is used as the value for the pool layer (i.e., average pooling). Other types of pooling may also be used and pooling layers may include any size and/or dimensionality. Therefore, the inclusion of the pooling layer reduces the size of the image that is input to the Inception-ResNet blocks, resulting in a reduced memory footprint. In some embodiments, the pooling layer is a max pooling layer, and the size of the encoded representation is 32×32×16×256, which is further fed into a global average pooling layer to further reduce its size. In other embodiments, other pooling layers and/or sizes may be used as aspects of the technology described herein are not limited in this respect.

FIG. 6B illustrates an exemplary architecture of an Inception-ResNet block 621, in accordance with some embodiments. The Inception-ResNet block 621 includes multiple convolutional layers and residual connections. The residual connections add the output of the convolutional layers to the convolution layer input 618, which may improve the training and performance of deep networks to increase the richness of the learned representations.

As described above in connection with FIG. 4, the feature extractor facility 600 may be trained using a collection of MRIs depicting ACLs. The trained feature extractor facility 600 may be used to further train a classifier, such as ACL classification module 410 and full knee classification module 420, for ACL injury classification (e.g., intact vs. injured), in accordance with some embodiments.

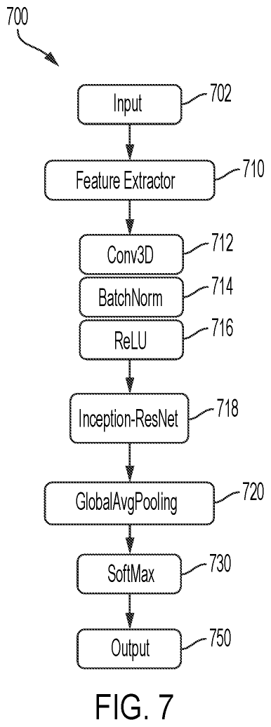

FIG. 7 illustrates an exemplary diagram of an ACL classifier for use in an ACL classification module, in accordance with some embodiments. ACL classifier 700 includes a trained feature extractor 710. Feature extractor 710 may be implemented using feature extractor 600, as described above with reference to FIG. 6A. However, in other embodiments, other implementations may be used as an addition to or alternate to feature extractor 600. The feature extractor 710 may be used as feature extractor in building a model for ACL injury risk prediction by adding several new layers on top of the feature extractor 710. These additional layers may include a convolutional layer 712, normalization 714, and ReLu layer 716 followed by an Inception-ResNet block 718. In some embodiments, the final part of this classifier is a Softmax layer 730 that outputs a binary probability vector for Intact versus Injured ACL classifications. The injury label was based on the ACL related adverse events with MRI and non-imaging data preceding any ACL related adverse event (e.g., ACL tear, ACL partial tear, ACL sprain, ACL laxity, anterior knee instability and excessive anterior knee laxity confirmed ACL tear) being marked as injured.

For training of this illustrative model, the layers that were previously optimized as part of the feature extractor network training were kept fixed, in accordance with some embodiments. The new layers are trained based on binary labels of intact versus injured. Given the class imbalance between Healthy and Injured classes, rather than using the cross-entropy loss the Tversky loss may be used for training. This loss may be more effective for applications where the data samples with positive labels account for a small fraction of the available training data. In this example, this loss is defined as:

$$loss = 1 - \frac{TP + 1}{TP + α FN + ( 1 - α ) FP + 1}$$

where TP=Σᵢlᵢ·q_{θ}(lᵢ|x), FN=Σᵢlᵢ·[1−q_{θ}(lᵢ|x)], and FP=Σᵢ(1−lᵢ)·q_{θ}(lᵢ|x) represent true positive rate, false negative rate and false positive rate, respectively; i∈{0,1} indicates label index of Healthy and Injured AIL; lᵢ∈{0,1} denotes ground-truth label; and q_{θ}(lᵢ|x)∈[0,1] demonstrates the CNN-predicted probability of label lᵢ with network parameter θ. To optimize the network parameters θ in this step, the Adam optimizer may be used with a learning rate of 10⁻⁵, in accordance with some embodiments.

For training an ACL classification module for ACL injury classification, segmented 3D MR images of intact ACLs and injured ACLs were used as training data, in accordance with some embodiments. For example, MR images were labeled as injured ACLs if they corresponded to the surgically treated ACLs of patients that experienced subsequent ACL related adverse events (i.e., ACL tear, ACL partial tear, ACL sprain, ACL laxity, anterior knee instability and excessive anterior knee laxity). As a result, the trained feature extractor may produce an ACL risk of injury sub-score.

A full knee classifier takes a knee MR image as input and outputs an ACL injury risk sub-score, which is the probability of the input image belonging to the Injured ACL class, in accordance with some embodiments. The architecture of the full knee classifier is similar to that of the feature extractor facility described above FIG. 6A. The feature extractor facility may be modified for use with a full knee 3D MR image. For example, the sizes of the input and output layers may be modified in order to accommodate the full knee MRI as input and output a two-class probability vector.

In some embodiments, the CNN parameters may be trained by first initializing the CNN parameters with the feature extractor model weights. The CNN parameters may then be fine-tuned for the new task using the Tversky loss function and the Adam optimizer with a learning rate of 10⁻⁶.

For training a full knee classification module, for ACL injury classification, full MR images of intact ACLs and injured ACLs were used as training data, in accordance with some embodiments. For example, MR images were labeled according to the same criteria as used in the ACL classification module. However, by training the full knee classification module, to classify surgically treated ACLs as intact or injured based on the entire knee MRI, separate from the ACL classification module, the full knee classification module may produce a risk of injury sub-score that may rely on features that were excluded from the ACL injury classification module. Thereby providing more accurate ACL injury classification, for some applications.

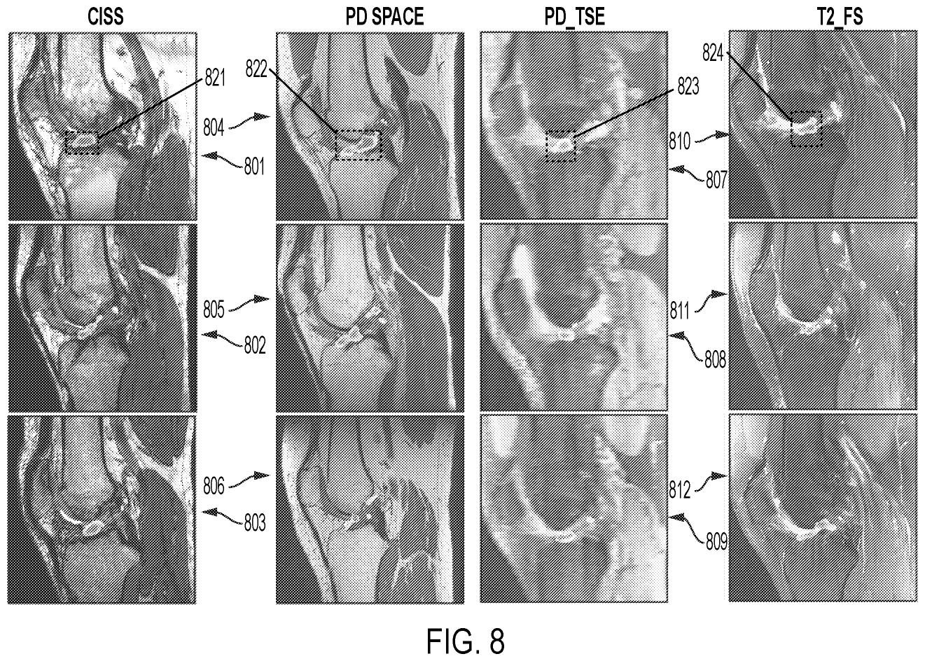

In some embodiments, occlusion maps may be generated to indicate the features associated with the injury classification as identified by the tissue condition determination facility to verify that the classification is based on relevant features and/or to identify potential imaging biomarkers. FIG. 8 illustrates several exemplary occlusion maps, in accordance with some embodiments. Occlusion maps were generated for true positive cases identified by the segmented ACL classifier to generate heatmaps around the features of the ACL which contributed to tissue condition determination facilities risk score. A representative set of MR images includes MR image 801, 802, 803, 804, 805, 806, 807, 808, 809, 810, 811, and 812. The MR images 801-812 illustrate the central slice of the ACL with a heatmap overlay indicating the features of the segmented ACL image that contributed to the correct decision by the classifier. MR images 801-812 arranged in rows show occlusion maps for the same knee under different MRI imaging sequences. Accordingly, MR images 801, 804, 807, and 810 correspond to the first knee; MR images 802, 805, 808, and 811 correspond to the second knee; and MR images 803, 806, 809, and 812 correspond to the third knee. MR images 801-812 arranged in columns show occlusions maps for different knees measured using the same MRI sequence. Accordingly, MR images 801, 802, and 803 correspond to MR images acquired using a constructive interference in steady state (CISS) sequence; MR images 804, 805, and 806 correspond to MR images acquired using a proton density (PD) sequence; MR images 807, 808, and 807 correspond to MR images acquired using a turbo spin echo (TSE) sequence; and MR images 810, 811, and 812 correspond to MR images acquired using a fat suppression (FS) sequence. As can be seen by comparing the MR images of the same knee under different pulse sequences in FIG. 8, different pulse sequences may result in different contrast between tissues and/or different degrees of sharpness in the image. For example, the MR images acquired using PD TSE sequences, such as images 807, 808, and 809 in FIG. 8, may appear less sharp than images acquired using other MRI sequences.

The color of the heat map corresponds to the relative positive contribution of the pixel to the classifiers ability to make the correct decision. In some embodiments, a red color may correspond to the highest positive contribution. As seen in the illustrated examples 801-812, the hot zone of the map, indicating features that contribute to the classification decision of injury, is consistently within the distal half of the ACL. This pattern is even consistent across different MRI sequences of the same subject, highlighting the tissue condition determination facility's ability to evaluate injury risk from MR images depicting an ACL independent of MRI sequence. For example, images 801, 804, 807, and 810 represent MR images of the same knee acquired using different pulse sequences with highlighted overlays corresponding to a tissue condition, the tissue condition being determined by the condition determination facility. Boxes 821, 822, 823, and 824 added to in images 801, 804, 807, and 810 respectively illustrate the consistent position of the highlighted tissue identified by the condition determination facility on the distal half of the ACL.

Techniques operating according to the principles described herein may be implemented in any suitable manner. Included in the discussion above are a series of flowcharts showing the steps or acts of various processes that analyze MR data of a joint to determine the condition of a tissue that may be used by a clinician or researcher to determine tissue health, strength, remodeling, risk of injury, and/or recovery after injury and treatment for a patient. The processing and decision blocks of the flowcharts above represent steps and acts that may be included in algorithms that carry out these various processes. Algorithms derived from these processes may be implemented as software integrated with and directing the operation of one or more single-or multi-purpose processors, may be implemented as functionally-equivalent circuits such as a Digital Signal Processing (DSP) circuit or an Application-Specific Integrated Circuit (ASIC), or may be implemented in any other suitable manner. It should be appreciated that the flowcharts included herein do not depict the syntax or operation of any particular circuit or of any particular programming language or type of programming language. Rather, the flowcharts illustrate the functional information one skilled in the art may use to fabricate circuits or to implement computer software algorithms to perform the processing of a particular apparatus carrying out the types of techniques described herein. It should also be appreciated that, unless otherwise indicated herein, the particular sequence of steps and/or acts described in each flowchart is merely illustrative of the algorithms that may be implemented and can be varied in implementations and embodiments of the principles described herein.

Accordingly, in some embodiments, the techniques described herein may be embodied in computer-executable instructions implemented as software, including as application software, system software, firmware, middleware, embedded code, or any other suitable type of computer code. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner. including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software clement. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques described herein may together form a complete software package. These functional facilities may, in alternative embodiments, be adapted to interact with other, unrelated functional facilities and/or processes, to implement a software program application, for example as a software program application such as a tissue condition determination facility.

Some exemplary functional facilities have been described herein for carrying out one or more tasks. It should be appreciated, though, that the functional facilities and division of tasks described is merely illustrative of the type of functional facilities that may implement the exemplary techniques described herein, and that embodiments are not limited to being implemented in any specific number, division, or type of functional facilities. In some implementations, all functionality may be implemented in a single functional facility. It should be appreciated that, in some implementations, some of the functional facilities described herein may be implemented together with or separately from others (i.e., as a single unit or separate units), or some of these functional facilities may not be implemented.

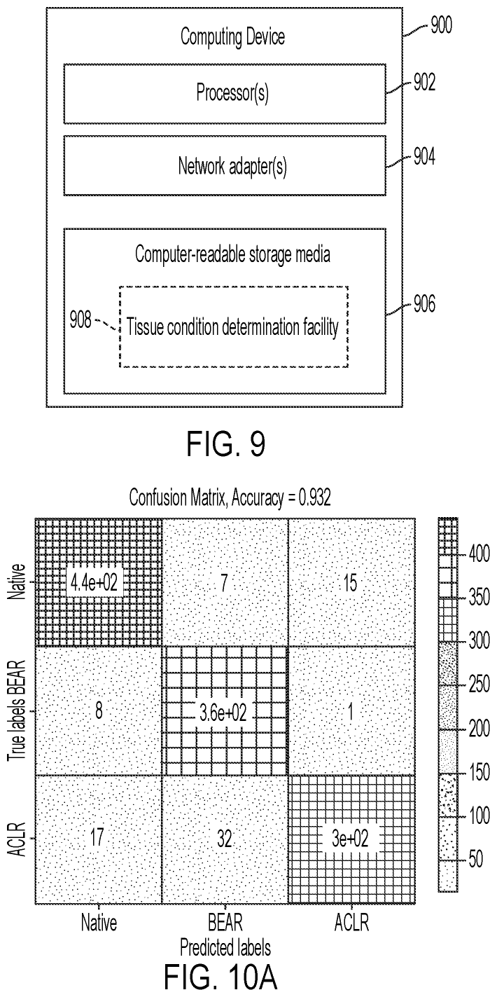

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as Compact Disk (CD) or a Digital Versatile Disk (DVD), a persistent or non-persistent solid-state memory (e.g., Flash memory, Magnetic RAM, etc.), or any other suitable storage media. Such a computer-readable medium may be implemented in any suitable manner, including as computer-readable storage media 906 of FIG. 9 described below (e.g., as a portion of a computing device 900) or as a stand-alone, separate storage medium. As used herein, “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

In some, but not all, implementations in which the techniques may be embodied as computer-executable instructions, these instructions may be executed on one or more suitable computing device(s) operating in any suitable computer system, including the exemplary computer system of FIG. 1, or one or more computing devices (or one or more processors or one or more computing devices) may be programmed to execute the computer-executable instructions. A computing device or processor may be programmed to execute instructions when the instructions are stored in a manner accessible to the computing device or processor, such as in a data store (e.g., an on-chip cache or instruction register, a computer-readable storage medium accessible via a bus, a computer-readable storage medium accessible via one or more networks and accessible by the device/processor, etc.). Functional facilities comprising these computer-executable instructions may be integrated with and direct the operation of a single multi-purpose programmable digital computing device, a coordinated system of two or more multi-purpose computing device sharing processing power and jointly carrying out the techniques described herein, a single computing device or coordinated system of computing devices (co-located or geographically distributed) dedicated to executing the techniques described herein, one or more Field-Programmable Gate Arrays (FPGAs) for carrying out the techniques described herein, or any other suitable system.

FIG. 9 illustrates one exemplary implementation of a computing device in the form of a computing device 900 that may be used in a system implementing embodiments of the techniques described herein, although others are possible. It should be appreciated that FIG. 9 is intended neither to be a description of necessary components for a computing device to operate as a tissue condition determination facility in accordance with the principles described herein, nor a comprehensive depiction.

Computing device 900 may comprise at least one processor 902, a network adapter 904, and computer-readable storage media 906. Computing device 900 may be, for example, a desktop or laptop personal computer, a personal digital assistant (PDA), a smart mobile phone, or any other suitable computing device. Network adapter 904 may be any suitable hardware and/or software to enable the computing device 900 to communicate wired and/or wirelessly with any other suitable computing device over any suitable computing network. The computing network may include wireless access points, switches, routers, gateways, and/or other networking equipment as well as any suitable wired and/or wireless communication medium or media for exchanging data between two or more computers, including the Internet. Computer-readable media 906 may be adapted to store data to be processed and/or instructions to be executed by processor 902. Processor 902 enables processing of data and execution of instructions. The data instructions may be stored on the computer-readable storage media 906.

The data and instructions stored on computer-readable storage media 906 may comprise computer-executable instructions implementing techniques which operate according to the principles described herein. In the example of FIG. 9, computer-readable storage media 906 stores computer-executable instructions implementing various facilities and storing various information as described above. Computer-readable storage media 906 may store tissue condition determination facility 908 configured to derive information indicative of a condition of a patient from MR data.

While not illustrated in FIG. 9, a computing device may additionally have one or more components and peripherals, including input and output devices. These devices can be used, among other things, to present a user interface. Examples, of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computing device may receive input information through speech recognition or in other audible format.

### EXAMPLES

An exemplary tissue condition determination facility to predict the risk of ACL injury based on a combination of imaging and non-imaging outcomes was developed and evaluated for accuracy, sensitivity, and specificity. A CNN classifier to study structural features of the different types of ACL tissues (i.e., native and surgically repaired or reconstructed) using a battery of isolated ACL segmentations was trained and validated. The CNN classifier was used as a feature extractor to develop and validate two additional CNN classifiers to predict risk of ACL injury using either segmented ACLs from MRI or whole knee MR images. The performance of different combinations of imaging (i.e., segmented ACL and whole knee MRI) and non-imaging outcomes were each tested to train and validate a multi-modal model that can predict the ACL injury risk. A multi-modal model is a model that has at least two inputs which are separately classified, where the two inputs correspond to different classifiers, and where the results of each classification are both used to determine an output. The models' performances were assessed based on overall accuracy, sensitivity and specificity, along with areas under the receiver operating characteristic and precision-recall curves. Occlusion maps were generated to better understand the features that model used to assign the classifications. The occlusion maps as well as standard performance metrics (i.e., accuracy, AUROC and AUPRC) were used to assess if the MRI sequence impacted the models' ability to predict the risk of ACL injury. All endpoints were selected prior to data collection.

The CNN feature extractor was trained using a collection of MRIs depicting ACLs. The MRIs were collected from surgically treated and contralateral knees of patients recruited in IRB and FDA approved BEAR trials and were manually segmented prior to training the CNN. The manually segmented ACLs were then augmented (i.e., translation, rotation, noise and blur) to generate a balanced distribution of all ACL types. The distribution of subjects and imaging data between classes, and test and training data sets is described in Table 1 below. The combination of raw and augmented segmentations was then randomly divided into training (82%) and validation (19%). This split was stratified by subject to ensure that the subjects from the validation set were completely unseen to the model, and the model would not be evaluated by subjects from the training set.

Segmented ACLs were used as inputs instead of the full knee MR images to train the CNN feature extractor to identify structural features of the ACL without being influenced by bold features existing in the tissue surrounding the ACL (e.g., bone tunnels in surgically treated ACLs). The trained CNN feature extractor achieved a classification accuracy of 93.2% (91.7%-94.7%), area under the receiver operating characteristic curve (AUROC) of 0.99 (0.98-0.99), and area under the precision-recall curve (AUPRC) of 0.98 (0.97-0.99) on the validation set. The trained feature extractor identified the type of ACLs in the validation set with 94.6% sensitivity and 96.0% specificity.

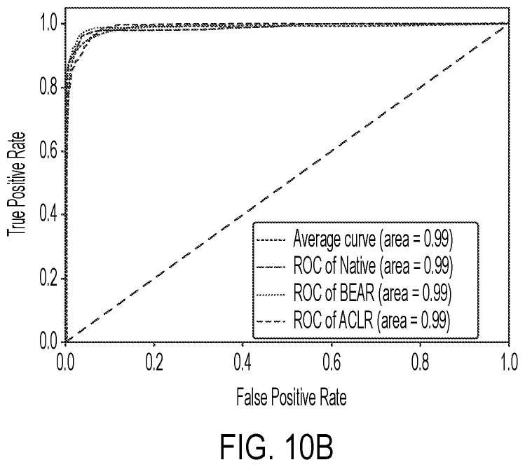

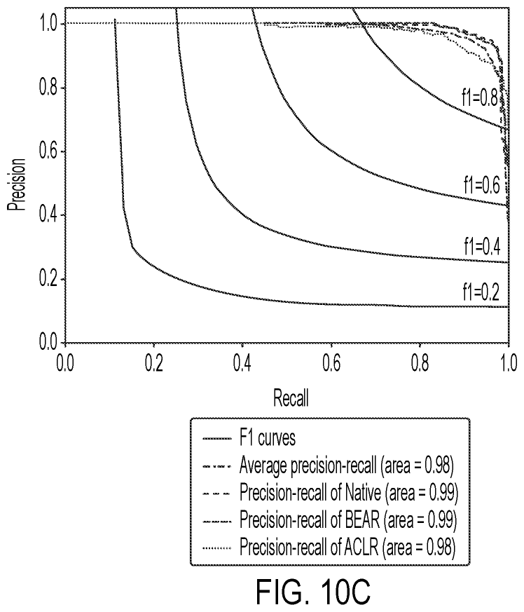

FIG. 10A illustrates a confusion matrix corresponding to the feature extractor performance in classifying ACL type from a segmented ACL, based on the validation set. FIG. 10B illustrates a receiver operating characteristic (ROC) curve corresponding to the feature extractor performance in classifying ACL type from a segmented ACL, based on the validation set. The ROC curves represent the diagnostic performance of a classifier by plotting the cumulative distribution function of the true positive rate (on the y-axis) versus the cumulative distribution function of the false positive rate (on the x-axis). A diagonal line is included with the plot to illustrate the diagnostic performance of randomly guessing the classification. FIG. 10C illustrates a precision recall curve corresponding to the feature extractor performance in classifying ACL type from a segmented ACL, based on the validation set. The precision recall curves plot the precision along the y-axis and recall along the x-axis of each classifier. The precision value represents the ratio of true positive to total positive results, including true positives and false positives. The recall value represents the ratio of the true positives to total positive samples, including true positives and false negatives. F1-scores are another metric for evaluating the performance of classification models. F1-scores represent the harmonic mean of the precision and the recall. FIG. 10C illustrates F1 curves along which the F1-score is constant as a function of the precision and recall values.

To further gauge the CNN feature extractor performance, a subset of 84 ACL segmentations from the validation set was randomly selected and deidentified to be reviewed by three experienced orthopedic surgeons involved in BEAR trials to independently identify the ACL type from ACL structure alone. All surgeons classified the same ACLs, but the orders were randomized between the examiners.

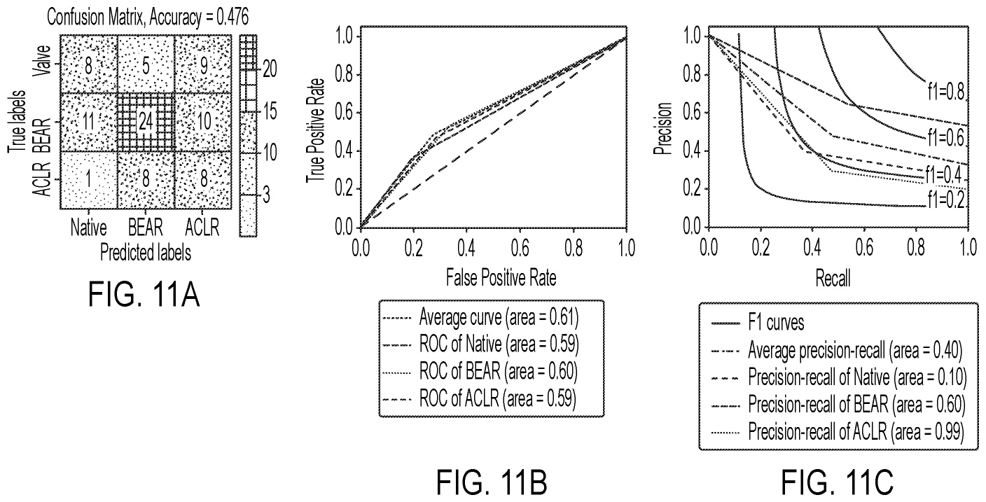

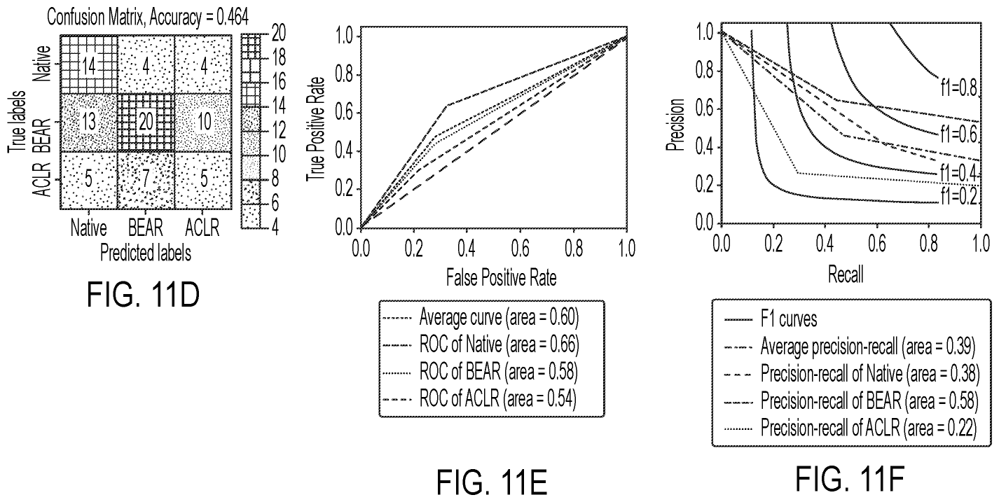

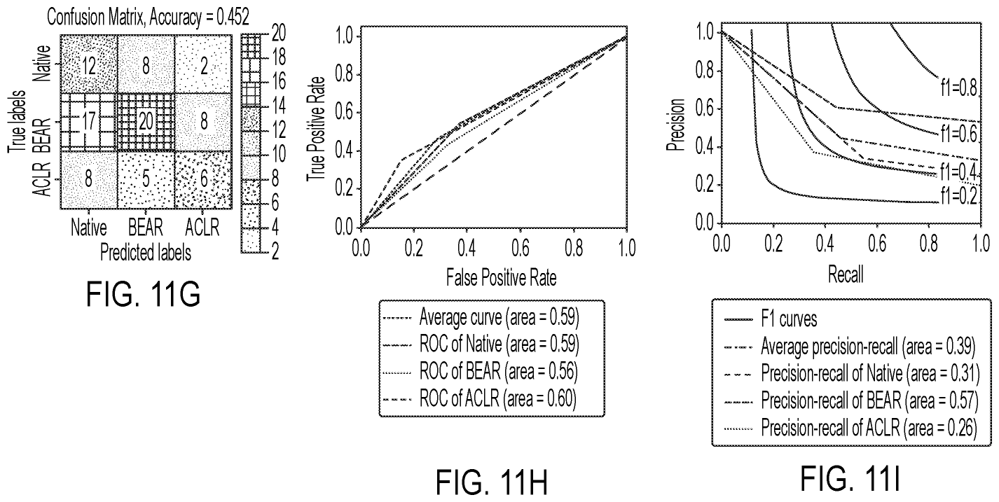

FIGS. 11A-11I illustrates human performance in classifying ACL type from segmented ACL. FIG. 11A illustrates a confusion matrix corresponding to the first human examiner. FIG. 11B illustrates a ROC curve corresponding to the first human examiner. FIG. 11C illustrates a precision recall curve corresponding to the first human examiner.

FIG. 11D illustrates a confusion matrix corresponding to the second human examiner. FIG. 11E illustrates a ROC curve corresponding to the second human examiner. FIG. 11F illustrates a precision recall curve corresponding to the second human examiner. FIG. 11G illustrates a confusion matrix corresponding to the third human examiner. FIG. 11H illustrates a ROC curve corresponding to the third human examiner. FIG. 11I illustrates a precision recall curve corresponding to the third human examiner.

As can be seen from comparing the human performances in classifying ACL type from segmented ACL (FIGS. 11A-11I and the performance of the CNN feature extractor in FIGS. 10A-10C, the CNN feature extractor outperformed the human examiners on all quantified performance metrics.

TABLE 1
Distribution of subjects and imaging data between classes, and test and training sets.

| | | | Raw Data Sample | | | Augmented Data Sample | | |
|---|---|---|---|---|---|---|---|---|
| | | | Distribution | | | Distribution | | |
| | | | Train | Test | Total | Train | Test | Total |
| Number of Subjects | | | 131 | 28 | 159 | 131 | 28 | 159 |
| Number | Ligament Type | Native | 196 | 58 | 254 | 1568 | 464 | 2032 |
| of MR | Classification | | | | (14%) | | | (33%) |
| Images | | BEAR | 813 | 182 | 994 | 1624 | 364 | 1988 |
| | | | | | (56%) | | | (33%) |
| | | ACLR | 438 | 76 | 514 | 1752 | 304 | 2056 |
| | | | | | (30%) | | | (34%) |
| | | Total | 1446 | 316 | 1762 | 4944 | 1132 | 6076 |
| | | | (82%) | (18%) | | (81%) | (19%) | |
| | ACL Injury | Intact | 1141 | 268 | 1409 | 2282 | 536 | 2818 |
| | Classification | | | | (80%) | | | (49%) |
| | | Injured | 292 | 61 | 353 | 2336 | 549 | 2885 |
| | | | | | (20%) | | | (51%) |
| | | Total | 1433 | 329 | 1762 | 4618 | 1085 | 5073 |
| | | | (81%) | (19%) | | (81%) | (19%) | |

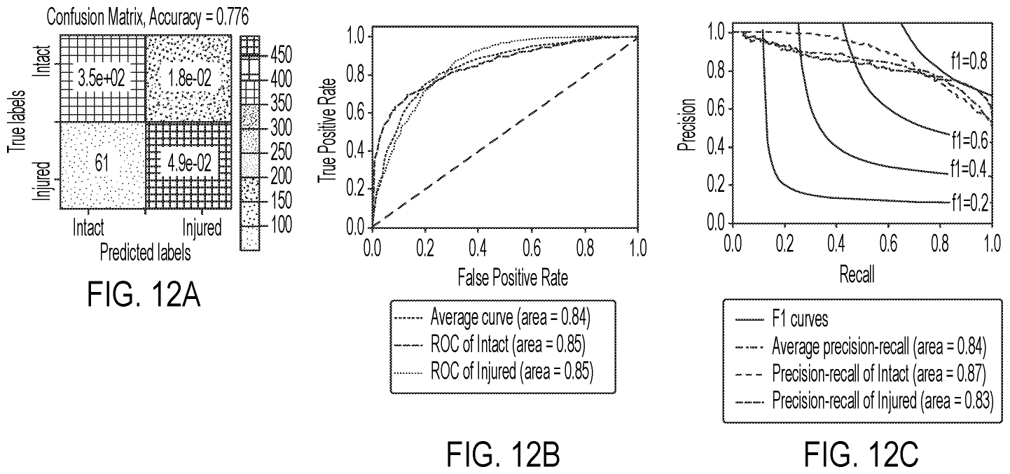

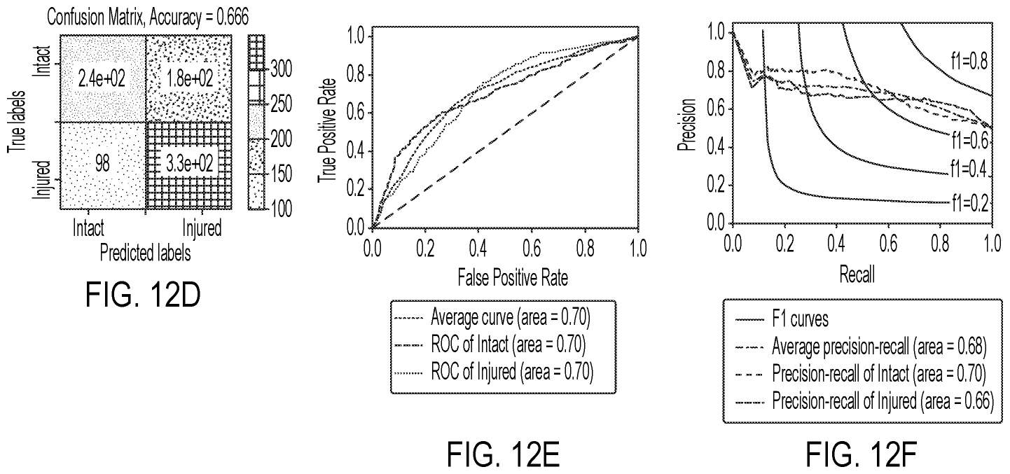

The pretrained network, as described above, was used as a feature extractor model to develop and train two CNN classifiers to predict risk of reinjury (i.e., intact vs injured). The first CNN classifier was trained and evaluated by the same group of manually segmented ACL MRIs that were used to train the feature extractor network. The distribution of subjects and imaging data between classes, and test and training data sets is reflected in Table 1. The segmented ACLs were annotated as either intact or injured. The manually segmented ACLs 10 were then augmented (i.e., translation, rotation, noise and blur) to generate a balanced distribution across intact and injured groups. The combination of raw and augmented segmentations was then randomly divided into training (81%) and validation sets (19%; Table 1). This split was stratified by subject to ensure that the subjects from the validation set were completely unseen to the model, and the model would not be evaluated using subjects from the training set. The trained feature extractor achieved a classification accuracy of 77.6%, (75.2%-80.1%), AUROC value of 0.84 (0.82-0.86), and AUPRC values of 0.84(0.81-0.87) on the validation set, as shown in FIGS. 12A-12C. The trained CNN was able to identify injured ACLs with 75.3% sensitivity and 80.6% specificity.

FIG. 12A illustrates a confusion matrix corresponding to the performance of the CNN trained using segmented ACL MRIs in identifying intact and injured ACLs. FIG. 12B illustrates a receiver operating characteristic (ROC) curve corresponding to the performance of the CNN trained using segmented ACL MRIs in identifying intact and injured ACLs. FIG. 12C illustrates a precision recall curve corresponding to a CNN classifier, trained using segmented ACL MRIs, in predicting the risk of injury based on segmented ACL.

To evaluate how other structural features of the knee joint influence injury risk prediction, a second CNN classifier was developed to identify knees with subsequent ACL injury from full knee MRIs. This second CNN classifier takes a 3D knee MR image stack as an input and outputs an estimated probability distribution over intact and injured ACL. The training and validation datasets were split and augmented in the same manner as described for the first CNN classifier of ACL injury as reflected in Table 1. The native ACLs were removed as they did not have any surgery related landmarks (e.g., bone tunnels) that could be precepted as features, contributing to risk of injury, by the CNN. The second CNN classifier resulted in a classification accuracy of 66.6% (63.8%-69.4%), AUROC value of 0.70 (0.67-0.73) and AUPRC value of 0.68 (0.64-0.72) on independent validation set, as shown in FIGS. 12D-12F. The trained CNN was able to identify injured ACLs with 68.7% sensitivity and 65.4% specificity.

FIG. 12D illustrates a confusion matrix corresponding to the performance of the CNN trained using full knee MRIs in identifying intact and injured ACLs. FIG. 12E illustrates a receiver operating characteristic (ROC) curve corresponding to the performance of the CNN trained using full knee MRIs in identifying intact and injured ACLs. FIG. 12F illustrates a precision recall curve corresponding to the CNN classifier, trained using full knee MRIs, in predicting the risk of injury based on segmented ACL.

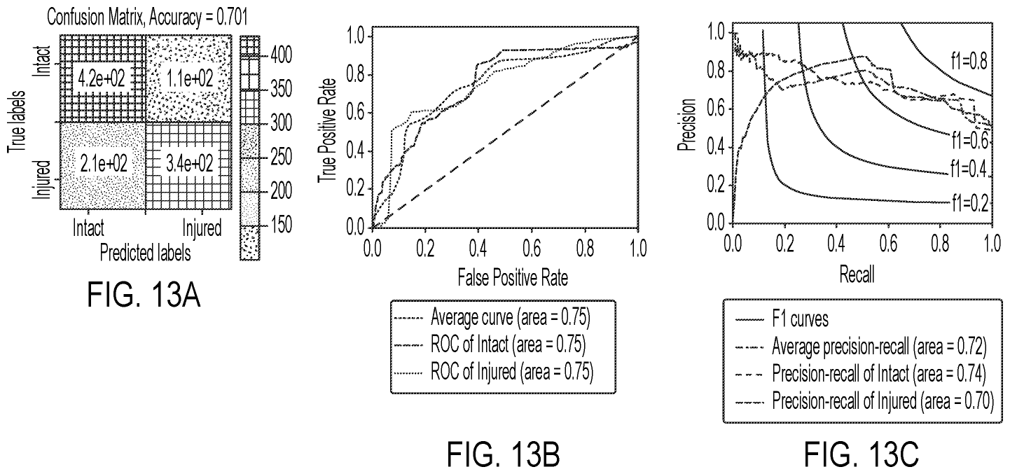

To further investigate how non-imaging factors may influence the performance of the CNN classifiers, a tissue condition determination facility that fused the segmented ACL and full knee CNNs with non-imaging clinical data to predict the risk of ACL injury was developed. The facility was designed using 11 non-imaging outcomes that are commonly recorded during postoperative visits that may be related to ACL function and biomechanics. The non-imaging data were then split to 81% training and 19% validation, as reflected in Table 1, to train and validate a logistic regression-based model to predict the risk of injury. The choice of logistic regression was based on series of analyses to look into the model's performance to identify injured and intact cases based on non-imaging data. The model achieved a classification accuracy of 70.1% (67.4%-72.8%), AUROC value of 0.75 (0.72-0.78) and AUPRC value of 0.72 (0.69-0.76) on the validation set as shown in FIGS. 13A-13C. The trained model was able to identify injured ACLs with 69.0% sensitivity and 71.4% specificity.

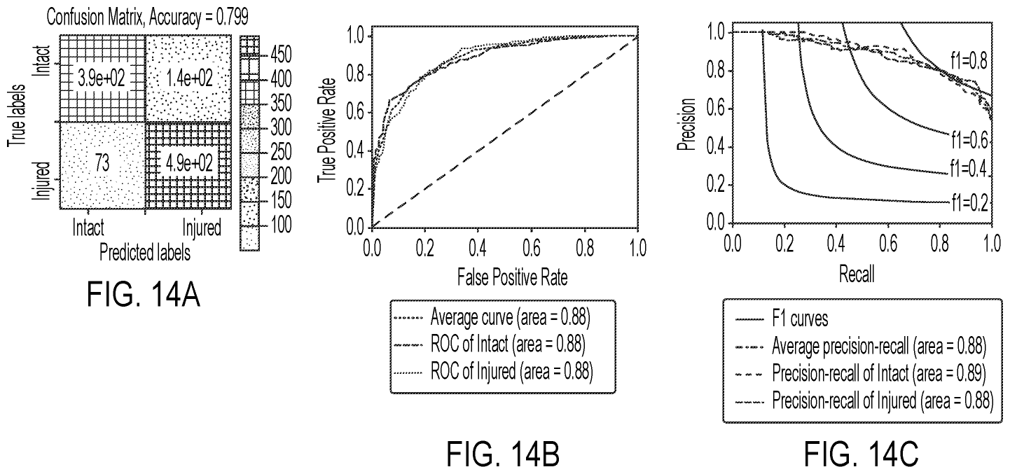

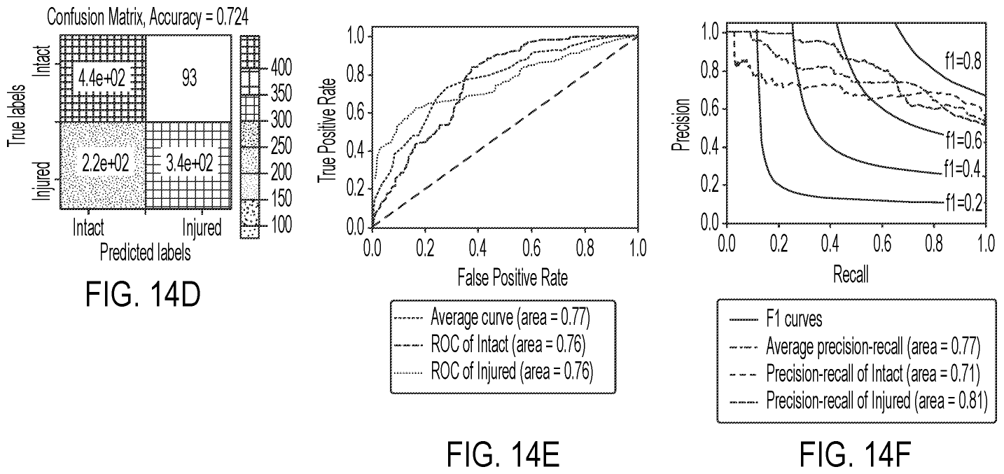

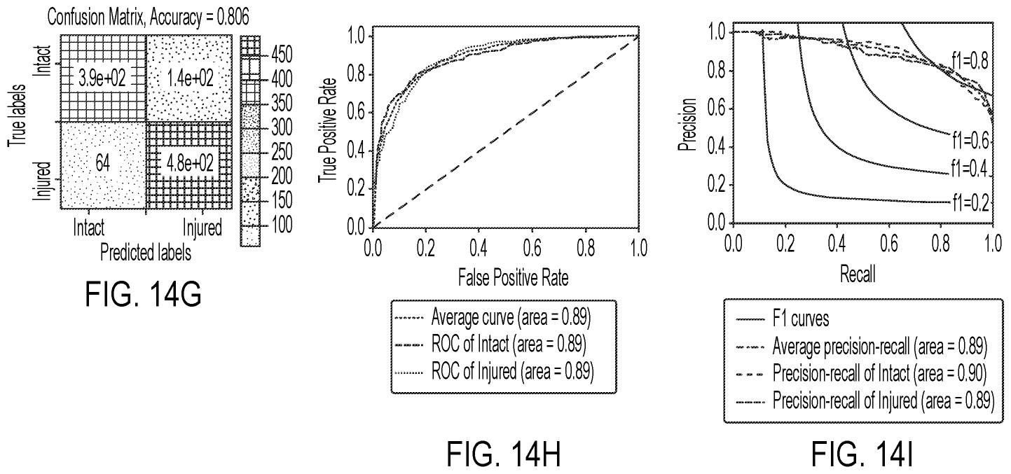

FIG. 13A illustrates a confusion matrix corresponding to the performance of the logistic regression-based model trained using non-imaging clinical data in identifying intact and injured ACLs. FIG. 13B illustrates a receiver operating characteristic (ROC) curve corresponding to the performance of the logistic regression-based model trained using non-imaging clinical data in identifying intact and injured ACLs. FIG. 13C illustrates a precision recall curve corresponding to the logistic regression-based model trained using non-imaging clinical data in predicting risk of injury based on non-imaging clinical data. In order to evaluate various combinations of the classifiers in the performance of the tissue condition determination facility structure in predicting the injury risk, three different fusion strategies were evaluated. In the first fusion strategy, the probability of injury risk obtained from the segmented ACL CNN classifier with those from non-imaging classifier were fused. FIG. 14A illustrates a confusion matrix corresponding to the performance of the first fusion strategy in identifying intact and injured ACLs. FIG. 14B illustrates a ROC curve corresponding to the performance of the first fusion strategy in identifying intact and injured ACLs. FIG. 14C illustrates a precision recall curve corresponding to the first fusion strategy in predicting risk of injury.

This first fusion strategy classification accuracy of 79.9% (77.5%-82.3%), AUROC value of 0.88 (0.86-0.90) and AUPRC value of 0.88 (0.85-0.90) on the validation set as shown in FIGS. 14A-14C. The fusion of segmented ACL CNN and non-imaging classifiers was able to identify injured ACLs with 80.0% sensitivity and 80.4% specificity.

The second fusion strategy included the whole knee MRI CNN and non-imaging classifier. FIG. 14D illustrates a confusion matrix corresponding to the performance of the second fusion strategy in identifying intact and injured ACLs. FIG. 14E illustrates a ROC curve corresponding to the performance of the second fusion strategy in identifying intact and injured ACLs. FIG. 14F illustrates a precision recall curve corresponding to the second fusion strategy in predicting risk of injury.

The second fusion strategy resulted in classification accuracy of 72.4% (69.7%-75.1%), AUROC value of 0.77 (0.74-0.80) and AUPRC value of 0.77 (0.74-0.81) on the validation set as shown in FIGS. 14D-14F. This model was able to identify injured ACLs with 72.4% sensitivity and 72.5% specificity.

The third fusion strategy included the segmented ACL CNN, full knee CNN, and non-imaging classifier. FIG. 14D illustrates a confusion matrix corresponding to the performance of the third fusion strategy in identifying intact and injured ACLs. FIG. 14E illustrates a ROC curve corresponding to the performance of the third fusion strategy in identifying intact and injured ACLs. FIG. 14F illustrates a precision recall curve corresponding to the third fusion strategy in predicting risk of injury.

The third fusion strategy resulted in highest classification accuracy (80.6% (78.2%-83.0%)), AUROC value (0.89 (0.87-0.91)), AUPRC value (0.89 (0.86-0.92)), and was able to identify injured ACLs with 77.2% sensitivity and 85.6% specificity as shown in FIGS. 14G-14I.

The input vectors of combinations of estimated risk sub-scores were labeled as Intact vs Injured according to the ground truth for each data set, and then fed into the model along with the corresponding labels as training and testing data. The output of the model is a single estimated risk score for ACL injury that is merged from the different data sources. The above fusion models were performed using a Logistic Regression classifier, within the same the limited-memory Broyden-Fletcher-Goldfarb-Shanno (LBFGS) algorithm minimizing cross-entropy loss. The choice of Logistic Regression to fuse the risk sub-scores is based on its performance compared to other approaches to distinguish between intact and injured cases.

While the non-imaging classifier had higher accuracy, sensitivity and specificity than the whole knee MRI classifier, it had a less stable precision-recall curve, in particular in classifying injured cases. The CNN classifiers based on segmented ACL or whole knee MRI had higher rates of false positives than false negative, whereas the non-imaging CNN classifier resulted in lower false positive than false negatives. The fusion of MRI (i.e., segmented ACL or whole knee) classifier predictions with non-imaging classifier predictions improved the model accuracy, sensitivity and specificity by up to 7%, but reduced the false positive rates in both classifiers.

Additionally, fused models had more robust precision-recall curves compared to non-imaging classifiers. The multi-modal classifier that leveraged the inputs from all three sources achieved the highest performance in predicting the ACL injury risk. While the multi-modal classifier had substantially better performance compared to whole knee MRI classifier or non-imaging classifier (up to 10% improvement in performance metrics), it had slightly better accuracy, sensitivity and specificity compared to segmented ACL classifier (<5% improvements). However, the multi-modal classifier resulted in ˜25% reduction in false positive rate compared to segmented ACL classifier. These observations suggest that while a CNN classifier based on an isolated segmented ACL is able to predict the risk of subsequent ACL injury, addition of non-ACL features (i.e., whole knee MRI and non-imaging data) may improve the prediction, in some applications, by reducing false positives.

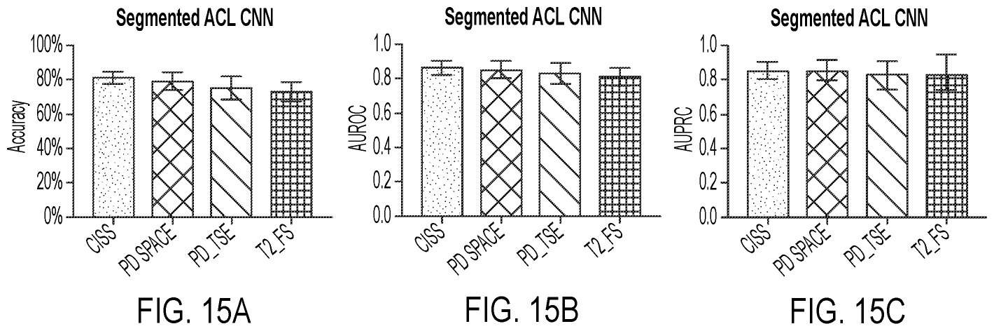

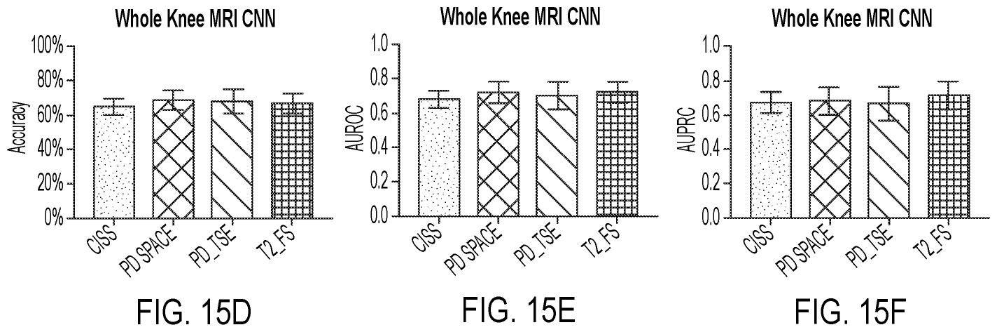

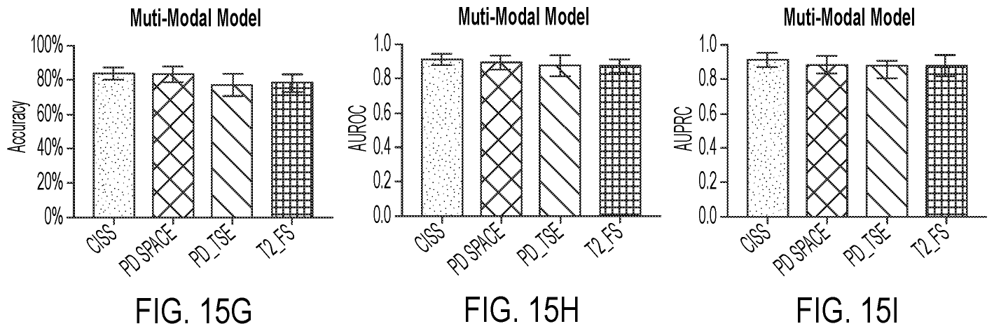

FIG. 15A-15I illustrates the classifiers' performance metrics for each independent tested MRI sequence. Performance metrics in predicted risk of ACL injury for CNN based on segmented ACL FIGS. 15A-15C, CNN based on whole knee MRI FIGS. 15D-15F, and Multi-modal model FIG. 15G-15I using segmented ACL, whole knee MRI, and non-imaging outcomes. The values are presented as mean (95% CI).

FIG. 15A illustrates the mean accuracy of the segmented ACL CNN for different MR imaging sequences. FIG. 15B illustrates the mean area under the ROC of the segmented ACL CNN for different MR imaging sequences. FIG. 15C illustrates the mean area under the PRC of the segmented ACL CNN for different MR imaging sequences. FIG. 15D illustrates the mean accuracy of the full knee MRI CNN for different MR imaging sequences. FIG. 15E illustrates the mean area under the ROC of the full knee CNN for different MR imaging sequences. FIG. 15F illustrates the mean area under the PRC of the full knee CNN for different MR imaging sequences. FIG. 15G illustrates the mean accuracy of the multi-modal model for different MR imaging sequences. FIG. 15H illustrates the mean area under the ROC of the multi-modal model for different MR imaging sequences. FIG. 15I illustrates the mean area under the PRC of the multi-modal model for different MR imaging sequences.

For all metrics, the confidence intervals are overlapping, indicating no significant differences between sequences. The similarities in model performance and occlusion maps between the sequences also illustrate that the tissue condition determination facility can predict the risk of ACL injury independent of the MRI sequence used to generate the MR image.

### Description of Exemplary ACL Treatment and Imaging

The unique and comprehensive data from 3 IRB and FDA approved clinical trials of ACL surgery (BEAR I: n=20, NCT02292004; BEAR II: n=100, NCT02664545; BEAR III: n=39; NCT03348995) were used. The cohort included 69 males and 90 females with an average age of 19.8±5.2 years (range: 14-36). All patients granted their informed consent prior to participating. All patients presented with a complete ACL tear, were less than 45 days from injury, had closed physes and who had at least 50% of the length of the ACL attached to the tibia (as determined from a pre-operative MR image). From 159 patients, 114 were treated bridge-enhanced ACL repair (BEAR) and 45 were treated with ACL reconstruction (ACLR) using hamstrings (n=43) or bone-patellar tendon-bone (n=2) autografts. Patients were excluded from enrollment if they had a history of prior ipsilateral knee surgery, history of prior knee infection, or had risk factors that could adversely affect ligament healing (nicotine/tobacco use, corticosteroids in the past six months, chemotherapy, diabetes, inflammatory arthritis). Patients were also excluded if they had a displaced bucket handle tear of the medial meniscus requiring repair. All other meniscal injuries were included. Patients were also excluded if they had a full thickness chondral injury, a grade III MCL injury, a concurrent complete patellar dislocation, or an operative posterolateral corner injury.

For ACL Reconstruction with Autograft Tendon (ACLR) procedures, a standard hamstring autograft procedure was performed using a quadruple semitendinosus-gracilis graft (n=33) or central third bone-patellar tendon-bone autograft (n=2) using a continuous-loop cortical button (Endobutton; Smith & Nephew, Andover, MA) for proximal fixation and a bioabsorbable interference screw (BioRCI HA; Smith & Nephew) for tibial fixation. A minimal notchplasty was performed at the surgeon's discretion as needed for adequate visualization of the posterior notch for placement of the femoral tunnel starting point within the prior ACL footprint. The femoral tunnel was drilled using an anteromedial portal technique and a flexible drill system (Clancy Anatomic Cruciate Guide, Smith & Nephew).

For Bridge-Enhanced ACL Repair (BEAR) procedures, an examination was performed to verify the positive pivot shift on the injured side and to record the Lachman test, range of motion and pivot shift exam results on both knees after the induction of general anesthesia. A tourniquet was then applied to the surgical limb. A knee arthroscopy was performed, and any meniscal injuries were treated if present. A tibial aimer (ACUFEX Director Drill Guide; Smith and Nephew, Andover, MA) was used to place a 2.4 mm guide pin through the tibia and the tibial footprint of the ACL. The pin was over-drilled with a 4.5 mm reamer (Endoscopic Drill; Smith & Nephew, Andover, MA). A notchplasty was performed using a combination of shaver and curette to facilitate visualization of the femoral footprint. A guide pin was then placed in the femoral ACL footprint, drilled through the femur and then over-drilled with the 4.5 mm reamer. A 4 cm arthrotomy was made at the medial border of the patellar tendon and a whip stitch of #2 absorbable braided suture (Vicryl; Ethicon, Cincinnati, OH) was placed into the tibial stump of the torn ACL. Two #2 non-absorbable braided sutures (Ethibond; Ethicon, Cincinnati OH) were looped through the two center holes of a cortical button (Endobutton; Smith & Nephew, Andover, MA). The free ends of a #2 absorbable braided suture from the tibial stump were passed through the cortical button, which was then passed through the femoral tunnel and engaged on the lateral femoral cortex. Both looped sutures of #2 non-absorbable braided (four matched ends) were passed through the scaffold, and 10 cc of autologous blood obtained from the antecubital vein was added to the scaffold. The scaffold was then passed up along the sutures into the femoral notch and the non-absorbable braided sutures were passed through the tibial tunnel and tied over a second cortical button on the anterior tibial cortex with the knee in full extension. The remaining pair of suture ends coming through the femur were tied over the femoral cortical button to bring the ACL stump into the scaffold using an arthroscopic surgeon's knot and knot pusher. The arthrotomy was closed in layers and the tourniquet deflated. Sterile dressings, followed by a cold therapy unit (Polar Care, Breg, Carlsbad, CA) and locking hinge knee brace (T-scope, Breg, Carlsbad, CA) were applied. No surgical drain was used.

For post-operative rehabilitation, a standardized physical therapy protocol, which did not specify which treatment the patient had received, was provided to all patients. The physical therapists were not informed of the treatment assignment of the patient. For all patients, a locking hinged brace (TScope; Breg, Carlsbad, CA) was applied to limit joint range of motion between 0 to 50 degrees of knee flexion for the first 2 weeks post-operatively, and from 0 to 90 degrees for the next four weeks unless they had a concomitant meniscal repair, in which case the brace range was restricted to 0 to 40 degrees for the first 4 weeks post-operatively before opening the brace up to 0 to 90 degrees of flexion. All patients were provided with a cold therapy unit (Iceman, DJO Global, Vista CA) for post-operative use. Both groups followed the same standardized physical therapy protocol including partial weight bearing for 2 weeks, then weight bearing as tolerated with crutches until 4 weeks post-operatively. Use of a functional ACL brace (CTi brace; OSSUR, Orange County, CA) was recommended from 6 to 12 weeks post-operatively and then for cutting and pivoting sports for 2 years after surgery. Other than the brace use and initial restricted weight bearing, the patients in both groups followed an identical rehabilitation protocol, adapted from that of the Multicenter Orthopaedic Outcomes Network (MOON). Phase 1 of the protocol emphasized reducing swelling and regaining full extension (weeks 0 to 2). Phase 2 (˜2 to 6 weeks) aimed at regaining quadriceps function and a normal gait pattern. Phase 3 (˜6 to 12 weeks) aimed at progressing neuromuscular function, and Phase 4 (˜12 to 18 weeks) focused on running and hopping. Running patterns and jumping began in Phase 5 (˜18 to 22 weeks), and patients were gradually progressed to sport specific skill training in Phase 6 (˜22 to 26 weeks). Patients were cleared for return to sport at the operating surgeon's discretion after completing an IKDC Subjective Score, hamstring and quadriceps strength measurement and bilateral hop testing at the 6-month visit.

TABLE 2
MR imaging acquisition parameters and distribution.

| | | Number of |
|---|---|---|
| Sequence | | Available |
| Name | Acquisition Parameters | MRI Sets |
| CISS | Sagittal Plane; TR/TE = 14/7 msec, FA = 35, 16 | 677 |
| | cm FOV, 80 × 512 × 512 (slice × frequency × | |
| | phase) | |
| PD | Sagittal Plane, TR/TE = 1000/49 msec, 16 cm | 405 |
| SPACE | FOV, 0.5 slice thickness and 320 × 320 (phase × | |
| | frequency) matrix | |
| PD-TSE | Coronal Plane, TR/TE = 1900/27 msec, 14 cm | 307 |
| | FOV, 3 slice thickness and 384 × 384 (phase × | |
| | frequency) matrix | |
| T2-FS | Axial Plane: TR/TE = 4300/83 msec, 16 cm | 373 |
| | FOV, 1 mm slice/gap, 256 × 256 matrix, | |

For acquiring MR images, patients underwent MR imaging of their surgically treated knee and contralateral side at multiple time points after ACL surgery. The scans occurred at 3, 6, 12 and 24 months in BEAR I trial, at 6, 12 and 24 months in BEAR II trial and at 9 months in BEAR III trial. Some patients also had additional scans in between their visits, as required by the clinical care team. Details of acquired sequences are presented in Table 2. All the scans were done with a single 3T scanner (Tim Trio, Siemens, Erlangen, Germany) and a 15-channel knee coil was used to scan the surgically treated and contralateral ACL-intact knees of each subject using common clinical sequences including Proton Density-weighted (PD-SPACE), T2 Fat-Saturation (T2-FS) and Proton Density-weighted Turbo Spin-Echo (PD-TSE) along with a 3D Constructive Interference in Steady State (CISS) sequence. The surgically treated ligaments and the contralateral native ACLs were manually segmented using image processing software (Mimics (v17.0; Materialize, Belgium)). The segmentation was done by an experienced investigator (AMK) with a high intra-rater reliability (ICC>0.9 for segmenting intact and surgically treated ACL).

Raw MR images were extracted from Digital Imaging and Communications in Medicine (DICOM) files, resampled to the same isotropic voxel size (0.5 mm×0.5 mm×0.5 mm) and then resized to 320×320×264 voxels. The manually segmented ACL masks were center-cropped into a size of 128×128×64. Raw MRIs were augmented with 1) addition of random Gaussian noise (with standard deviation in the range [0, 10]) to voxel signal intensities, 2) random Gaussian blur (with standard deviation in the range [0, 0.2]), 3) 3D rotation (with rotation angles around the X, Y, and Z axes chosen from a uniform distribution in the ranges [−5, 5] degrees), and 4) 3D translation (with translation along the X, Y, and Z axes chosen from a uniform distribution in the range [−10, 10] voxels). The augmentations occurred to either the ACL segmentation mask or to the entire stack of MRI image. Each augmented MR image was created by applying all four operations in sequence, every time choosing the augmentation parameters at random. The utilized augmentation techniques effectively increase the variation of raw MRI images, thereby imposing higher robustness to noise and variability of the system and helped with reducing the class imbalance and increase the sample size. The distribution of subjects and MR images between classes (i.e., ligament type, injury status), test and training sets are presented in Table 1 above.

### Exemplary Non-Imaging Outcomes

For collecting non-imaging outcomes, International Knee Documentation Committee (IKDC) Subjective Scores, instrumented AP knee laxity assessments, muscle strength assessments, knee range of motion measurements, and functional hop tests were collected in a clinical setting.

The IKDC Subjective Score is commonly used to assess patient-reported outcomes after ACL surgery. The form used for IKDC scoring assesses the patient's perception of knee function, symptoms, and sports performance.

The instrumented AP knee laxity assessment (Knee Arthrometry) was conducted using a compuKT 2000 knee arthrometer (MEDmetric Corp, San Diego, CA) to measure total translational motion between the tibia relative to the secured femur. Testing was be done according to the MOON protocol in compliance with the manufacturer's operation manual. Each leg placed on the adjustable thigh support with knee stabilized at 20° to 35° of flexion. The arthrometer was secured to the shank such that the patellar sensor pad is resting on the patella with the knee joint line reference mark on the arthrometer aligned with the subject's joint line. The ankle and foot were stabilized to limit leg rotation. The examiner applied a posterior (−134N) and anterior (+134N) pressure on an axis perpendicular to the tibia. Displacement (mm) was then documented. Each test was performed three times and the mean of the three tests was used to obtain a side-to-side difference between the treated and contralateral limbs. An independent examiner performed the tests and knee sleeves were used to cover both knees. The examiner was blinded to the surgical side and study group assignment when performing the physical examination.

Muscle strength assessments of the hamstring and quadriceps muscle isometric muscle strengths were measured using a hand-held dynamometer (Microfet 2; Hoggan Scientific, LLC, Salt Lake City, UT) that has specifically been validated as a reliable hand-held dynamometer (HHD). The hamstring strength was measured with the patient prone and the knee in 90° of flexion. The dynamometer was placed on the posterior surface of the lower leg proximal to the ankle. The quadriceps strength was measured with the knee at 90 degrees of flexion with the dynamometer positioned at the distal tibia. An independent examiner performed the tests and knee sleeves were used to cover both knees. The examiner was blinded to the surgical side and study group assignment when performing the physical examination.

Knee range of motion measurements of the knee passive and active range of motion were measured at each postoperative visit using a goniometer. Knee sleeves were used to cover both knees for all patients, and the examiner was blinded to the surgical side and study group when performing the physical examination. Measurements were done on both knees.

Functional hop tests were conducted with subjects performing single hop aiming for maximum hop distance and measuring the distance. The tests included triple and straight single leg hop, triple and diagonal single leg hop, and a 6-meter timed hop. All tests were performed bilaterally. All measures were performed in duplicate on each side and the duplicate measurements averaged for further analysis. An independent examiner performed the tests and knee sleeves were used to cover both knees. The examiner was blinded to the surgical side and study group assignment when performing the physical examination.

In addition to, clinical outcomes the age and gender were also collected. The collected non-imaging data for each patient visit were represented with a vector of 11 elements. Hence, each MR image has a corresponding vector of clinical data. To address missing data point, imputation may be used, which aims at inferring plausible values for the missing predictors. Based on preliminary cross-validation experiments, a median-based imputation approach was used, which resulted in higher classification accuracy compared to other imputers such as mean, constant value, and K nearest neighbors. The non-imaging data were then normalized by removing the mean and scaling to unit variance, separately for each of the 11 variables. The non-imaging training dataset was upsampled using an SVM-SMOTE algorithm to increase sample size and to balance classifications.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly. embodiments may be constructed in which acts are performed in an order different than illustrated, which may include performing some acts simultaneously, even though shown as sequential acts in illustrative embodiments.

Various aspects of the embodiments described above may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects describe in other embodiments.

Use of ordinal terms such as “first.” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phrascology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

The word “exemplary” is used herein to mean serving as an example, instance, or illustration. Any embodiment, implementation, process, feature, etc. described herein as exemplary should therefore be understood to be an illustrative example and should not be understood to be a preferred or advantageous example unless otherwise indicated.

Having thus described several aspects of at least one embodiment, it is to be appreciated that various alternations, modifications, and improvements will readily occur to those skilled in the art. Such alterations, modifications, and improvements are intended to be part of this disclosure and are intended to be within the spirit and scope of the principles described herein. Accordingly, the foregoing description and drawings are by way of example only.

What is claimed is:

1. A computer system, comprising:
a computer processor; and
a non-transitory computer-readable storage medium storing processor-executable instruction that, when executed by the processor, cause the processor to perform a method of determining a condition of a tissue of a patient, the method comprising:
determining, using a first trained statistical classifier, a first value indicative of a risk of injury of the tissue from a first magnetic resonance image depicting a tissue;
determining, using a second trained statistical classifier, a second value indicative of a risk of injury of the tissue from a second magnetic resonance image depicting the tissue and surrounding anatomy; and
determining a risk of injury score for the patient using the first value and the second value.

2. The system of claim 1, wherein the first magnetic resonance image depicting the tissue comprises a magnetic resonance image depicting a connective tissue of a knee of the patient.

3. The system of claim 2, wherein the first magnetic resonance image depicting the connective tissue comprises a magnetic resonance image depicting an Anterior Cruciate Ligament (ACL) of the patient, and wherein the second magnetic resonance image depicting the tissue and the surrounding anatomy comprises a magnetic resonance image depicting the knee of the patient.

4. The system of claim 3, wherein the first magnetic resonance image is generated from the second magnetic resonance image.

5. The system of claim 1, wherein the second trained statistical classifier uses patterns indicative of a risk for injury to determine the second value.

6. The system of claim 1, wherein the risk of injury is a risk of reinjury at a time after a ligament reconstruction surgery.

7. A method of determining a condition of a tissue of a patient, the method comprising:
determining a first value indicative of a risk of injury of the tissue from a first magnetic resonance image depicting a tissue;
determining a second value indicative of a risk of injury of the tissue from a second magnetic resonance image depicting the tissue and surrounding anatomy; and
determining a risk of injury score for the patient using the first value and the second value.

8. The method of claim 7, wherein the first magnetic resonance image depicting the connective tissue comprises a magnetic resonance image depicting an Anterior Cruciate Ligament (ACL) of the patient, and wherein the second magnetic resonance image depicting the tissue and the surrounding anatomy comprises a magnetic resonance image depicting the knee of the patient.

9. The method of claim 8, further comprising generating the first magnetic resonance image by segmenting a plurality of points from the second magnetic resonance, wherein segmenting a plurality of points comprises:
identifying points in the second magnetic resonance image associated with the tissue; and
generating an image including only the points associated with the tissue.

10. The method of claim 7, wherein determining a first value comprises a first trained statistical classifier determining the first value from the magnetic resonance image depicting the tissue of the patient.

11. The method of claim 10, wherein determining a second value comprises a second trained statistical classifier that determines the second value from the magnetic resonance image depicting the tissue and the surrounding anatomy of the patient.

12. The method of claim 11, wherein the second trained statistical classifier uses patterns indicative of a risk for injury to determine the second value.

13. The method of claim 11, wherein determining a risk of injury score further comprises using a third value indicative of a risk of injury from non-image data.

14. The method of claim 13, wherein the non-image data is clinical data of the patient.

15. The method of claim 13, wherein determining a third value comprises a third trained statistical classifier that determines the third value from non-image data of the patient.

16. The method of claim 15, wherein determining a risk for injury score comprises providing the first value, second value, and third value to a classifier that generates the risk for injury score.

17. The method of 15, further comprising:
training a first trained statistical classifier, wherein the training comprises training a neural network to classify images depicting tissue based on risk of injury, wherein training the neural network comprises performing unsupervised training using images depicting tissue; and
training a second trained statistical classifier, wherein the training comprises training a neural network to classify images depicting tissue and the surrounding anatomy based on risk of injury, wherein training the neural network comprises performing unsupervised training using images depicting tissue and the surrounding anatomy.

18. The method of 17, further comprising training the third trained statistical classifier, wherein the training comprises training a neural network to classify non-image data based on risk of injury, wherein training the neural network comprises performing unsupervised training using non-image data.

19. The method of claim 7, further comprising generating an annotated image, comprising:
determining locations in the first and second magnetic resonance image that contribute to an increased risk of injury;
visually annotating the first and/or second magnetic resonance image at locations that contribute to the increased risk of injury; and
outputting the annotated image.

20. At least one non-transitory computer-readable storage medium storing processor-executable instruction that, when executed by at least one processor, cause the at least one processor to perform a method comprising:
determining a first value indicative of a risk of injury of the tissue from a first magnetic resonance image depicting a tissue;
determining a second value indicative of a risk of injury of the tissue from a second magnetic resonance image depicting the tissue and surrounding anatomy; and
determining a risk of injury score for the patient using the first value and the second value.
